(19) United States
(12) Patent Application Publication
(10) Pub. No.: US 2018/0110938 A1
(43) Pub. Date: Apr. 26, 2018

(54) Method and Device for Vaporizing of Phyto Material

(71) Applicant: Michael Alexander Trzecieski, Mid Levels (HK)

(72) Inventor: Michael Alexander Trzecieski, Mid Levels (HK)

(21) Appl. No.: 15/453,001

(22) Filed: Mar. 8, 2017

Related U.S. Application Data
(63) Continuation-in-part of application No. 15/240,203, filed on Aug. 18, 2016.

(51) Int. Cl. A61M 11/04; A61M 21/02; A61B 34/30

(52) U.S. Cl. CPC A61M 11/042; A61M 2021/0016; A61B 34/30; A61M 21/02

(57) ABSTRACT

A novel device for vaporization device is disclosed for frictionally engaging a water pipe having an input port and an inhalation aperture with a water pipe fluid pathway formed therebetween comprising. The device includes a electronic vaporization element (EVE) formed from an elongated hollow member having a first end and a second end for coupling with a water pipe input port. A heating element is disposed proximate the first end and powered by a removable electrical power source for being coupled with the water pipe using a frictional engagement mechanism. A first control circuit is electrically coupled with the electrical power source for providing electrical power from the electrical power source to the heating element for heating the phyto material to a predetermined temperature for causing vaporization thereof.

### CROSS REFERENCE TO RELATED APPLICATION

This application is a continuation-in-part of U.S. patent application Ser. No. 15/240,203 filed Aug. 18, 2016, which is based on, and claims priority to, U.S. Provisional Application No. 62/215,168, filed on Sep. 8, 2015, and claims priority to U.S. Provisional Applications 62/455,174 filed on Feb. 6, 2017 and 62/460,875 filed on Feb. 20, 2017, the entireties of which are herein incorporated by reference.

### TECHNICAL FIELD OF THE INVENTION

The technical field relates to a device for vaporization of phyto materials and more specifically to a device for vaporization of phyto material and phyto material extracts.

### BACKGROUND OF THE INVENTION

Aromatherapy generally uses essential oils, which are extracted from phyto materials, such as leaves of plants, for therapeutic benefits. These essential oils are either massaged into the skin or can be inhaled. In some cases the phyto materials are heated in order to released the essential oils therefrom. By heating these phyto materials at predetermined temperatures, essential oils and extracts are boiled off, depending upon the temperature at which these phyto materials are heated, an aroma or vapor is given off, which is then inhaled by a user for its therapeutic benefits. Devices that provide such operation are generally known as vaporizers. There are also extracts available that are derived from the phyto material or loose-leaf aromatherapy materials and these have a consistency of honey and are typically highly purified forms. Normally these extracts are vaporized at temperatures between 500 to 700 degrees Fahrenheit. Phyto materials are normally vaporizer at 330 to 440 degrees Fahrenheit.

Devices that process these phyto material and phyto material extracts typically include a water pipe, or bong, or dab rig, that has an input port and an inhalation aperture with a fluid pathway formed therebetween a water trap disposed between the input port and the inhalation aperture as part of the fluid pathway. The water trap contains water and serves to filter incoming ambient air and phyto material extract vapor as it propagates therethrough. When a user inhales from the inhalation aperture, ambient air enters the input port and percolates through the water trap to be inhaled from the inhalation aperture. Water pipes are very well known in the art.

Normally a metal or ceramic electronic vaporization element is inserted into the input port and it is heated with a torch to get it to reach a temperature of about 500 to 700 degrees Fahrenheit. Measurement of the temperature of the electronic vaporization element is not measured and usually the process is a visual or time based one. Phyto material extract is applied to the electronic vaporization element and a user inhales from the inhalation aperture of the water pipe, which results in vaporized phyto material and ambient air to flow into the inhalation aperture and into the fluid pathway for being cooled by the water which is typically disposed within this fluid pathway to cool the vapor air mixture.

Because the heating is performed by a torch, such devices do not typically vaporize the phyto material extracts 419 and instead combust them. Heating to combustion temperatures usually results in smoke and other combustion by products to be inhaled from the inhalation aperture. This combustion of course isn't a safe process as there are many harmful byproducts released in the combustion process. Glass or ceramic electronic vaporization elements are preferable as these materials offer an experience that affects a taste of the vapor the least.

There are other solutions on the market that utilize a metal or element with a heater coil wrapped around it that are normally plugged into a wall, however these devices are cumbersome and not power efficient because of an amount of thermal mass that needs to be heated in order to attain a required vaporization temperature of the heated member. They are also not appealing in product design and can lead to end users tripper over the power supply cables. Not to mention that these devices are also not portable and when powered on and plugged in and potentially tipped over, may be a fire hazard.

It is therefore an object of the invention to provide an aromatherapy vaporization device that overcomes the aforementioned deficiencies.

### SUMMARY

In accordance with the embodiments of the invention there is provided a device for vaporization of phyto material extracts for attaching to a water pipe having an input port and an inhalation aperture with a water pipe fluid pathway formed therebetween comprising: a electronic vaporization element comprising: an elongated hollow member formed from a low thermal conductivity material having a first end and a second end opposite the first end, a fluid pathway propagating through the elongated hollow member from the first end to the second end thereof, the second end for coupling with the water pipe input port; an annular heating element having a first side and a second side opposite the first side, the annular heating element thermally coupled with the elongated hollow member proximate the first end and having the first side facing the first end with the fluid pathway propagating through a center thereof, the annular heating element comprising a first electrical contact and a second electrical contact proximate the second side, the annular heating element secured to the elongated hollow member for allowing thermal expansion thereof along a radial axis perpendicular to the fluid pathway, the annular heating element comprising a resistive heater disposed between the first and second electrical contacts and proximate the second side; and an electrical power source electrically coupled with the first and second electrical contacts for providing of electrical power to the resistive heater for heating of the resistive heater for imparting thermal energy to the annular heating element, wherein during heating of the resistive heater, a portion of the thermal energy is transferred to the annular heating element first side and another portion, other than the first portion, is transferred to the elongated hollow member proximate the first end, upon the annular heating element second side reaching a predetermined temperature the phyto material extract is applied to the annular heating element first side and becomes vaporized and upon inhalation from the inhalation aperture this vapor is mixed with ambient air and flows through the fluid pathway from the first end where it loses thermal energy to the elongated hollow member proximate the second end as it propagates through the input port of the water pipe and through the water pipe fluid pathway and to the inhalation aperture.

In accordance with the embodiments of the invention there is provided a device for vaporization of phyto material extracts for attaching to a water pipe having an input port and an inhalation aperture with a water pipe fluid pathway formed therebetween comprising: a electronic vaporization element comprising: an elongated hollow member formed from a low thermal conductivity material having a first end and a second end opposite the first end, a fluid pathway propagating through the elongated hollow member from the first end to the second end thereof, the second end for coupling with the water pipe input port; an annular heating element having a first side and a second side opposite the first side, the annular heating element thermally coupled with the elongated hollow member proximate the first end and having the first side facing the first end with the fluid pathway propagating through a center thereof, the annular heating element comprising a first electrical contact and a second electrical contact proximate the second side, the annular heating element secured to the elongated hollow member using silica and for allowing thermal expansion of the annular heating element along a radial axis perpendicular to the fluid pathway, the annular heating element comprising a metallic planar heater disposed on the second side between the first and second electrical contacts; an electrical power source comprising a plurality of batteries electrically coupled with a first control circuit, which is electrically coupled with the first and second electrical contacts for controllably providing of electrical power to the metallic planar heater for heating of the metallic planar heater for imparting thermal energy to the annular heating element, wherein during heating of the metallic planar heater, a portion of the thermal energy is transferred to the annular heating element first side and another portion, other than the first portion, is transferred to the elongated hollow member proximate the first end, upon the annular heating element second side reaching a predetermined temperature the phyto material extract is applied to the annular heating element first side and becomes vaporized and upon inhalation from the inhalation aperture this vapor is mixed with ambient air and flows through the fluid pathway from the first end where loses thermal energy to the elongated hollow member proximate the second end as it propagates through the input port of the water pipe and through to the water pipe fluid pathway and through the inhalation aperture; and a first housing for having the electrical power source contained there and the plurality of batteries, the first housing comprising an adjustable clamping mechanism for frictionally engaging of the water pipe.

In accordance with the embodiments of the invention there is provided a device for vaporization of phyto material extracts for attaching to a water pipe having an input port and an inhalation aperture with a water pipe fluid pathway formed therebetween comprising: a electronic vaporization element comprising: an elongated hollow member formed from a low thermal conductivity material having a first end and a second end opposite the first end, a fluid pathway propagating through the elongated hollow member from the first end to the second end thereof, the second end for coupling with the water pipe input port; a partial annular heating element radially disposed about the elongated hollow member, the partial annular heating element having a first side and a second side opposite the first side, the partial annular heating element thermally coupled with the elongated hollow member proximate the first end and having the first side facing the first end with the fluid pathway propagating through a center thereof, the partial annular heating element comprising a first electrical contact and a second electrical contact proximate the second side, the partial annular heating element secured to the elongated hollow member for allowing thermal expansion thereof along a radial axis perpendicular to the fluid pathway, the partial annular heating element comprising a resistive heater disposed between the first and second electrical contacts and proximate the second side; an electrical power source electrically coupled with the first and second electrical contacts for providing of electrical power to the resistive heater for heating of the resistive heater for imparting thermal energy to the partial annular heating element, wherein during heating of the resistive heater, a portion of the thermal energy is transferred to the partial annular heating element first side and another portion, other than the first portion, is transferred to the elongated hollow member proximate the first end, upon the partial annular heating element second side reaching a predetermined temperature the phyto material extract is applied to the partial annular heating element first side and becomes vaporized and upon inhalation from the inhalation aperture this vapor is mixed with ambient air and flows through the fluid pathway from the first end where loses thermal energy to the elongated hollow member proximate the second end as it propagates through the input port of the water pipe and through the water pipe fluid pathway and through to the inhalation aperture.

In accordance with the embodiments of the invention there is provided a method and device for vaporizing phyto material for frictionally engaging a water pipe having a water pipe input port and an inhalation aperture with a water pipe fluid pathway formed therebetween comprising: an electronic vaporization element (EVE) comprising: an elongated hollow member comprising a low thermally conductivity material having a first end and a second end opposite the first end, a fluid pathway propagating from the first end to the second end thereof, the second end for coupling with the water pipe input port, the elongated hollow member proximate the first end having a phyto material contact surface and having disposed opposite thereof a second side phyto material contact surface, the phyto material for being applied to the phyto material contact surface proximate the first end; a heating element comprising a first electrical contact and a second electrical contact and disposed proximate the first end and in proximity of the second side phyto material contact surface and opposite the phyto material contact surface, the heating element being partially disposed within a heating element housing; a frictional coupling formed between the heating element housing and the elongated hollow member proximate the first end for releasably coupling of the heating element with the elongated hollow member proximate the first end; the heating element for applying heat to the second side phyto material contact surface and for a portion of the applied heat to propagate through the elongated hollow member proximate the first end into the phyto material contact surface to which the phyto material is applied, the phyto material contact surface for heating of the phyto material by the propagated portion of the applied heat to a predetermined temperature for vaporizing of the phyto material for creating a vapor therefrom and upon inhalation from the inhalation aperture this vapor is mixed with ambient air and flows through the fluid pathway from the first end and propagates through the input port of the water pipe and through to the inhalation aperture; a removable electrical power source comprising a first housing for having an electrical power source contained therein, the first housing comprising a frictional engagement mechanism for frictionally engaging of the water pipe; and, a first control circuit disposed within the first housing and electrically coupled with the electrical power source and the first and second electrical contacts 108 of the EVE with an electronic vaporization element coupling cable electrically disposed therebetween, the first control circuit for providing electrical power from the electrical power source to the heating element for heating the phyto material to the predetermined temperature.

A method and device for vaporizing phyto material for frictionally engaging a water pipe having an input port and an inhalation aperture with a water pipe fluid pathway formed therebetween comprising: providing an electronic vaporization element comprising an elongated hollow member having a first end disposed proximate a heating element and a second end opposite the first end, a fluid pathway propagating from the first end to the second end thereof with the heating element 8806 disposed proximate the first end; coupling the EVE second end with the water pipe input port; providing a first housing for having an electrical power source contained therein and comprising a frictional engagement mechanism for releasably frictionally engaging the water pipe; frictionally engaging the water pipe with the frictional engagement mechanism for releasably coupling of the first housing to the water pipe; disposing phyto material extract proximate the heating element; heating of the phyto material extract to a predetermined temperature, where the predetermined temperature is a temperature that results in a vaporization of the phyto material; vaporizing of the phyto material extract for creating a vapor therefrom; and inhaling from the inhalation aperture and having the vapor mixing with ambient air for flowing through the fluid pathway from the first end through the second end and through the input port of the water pipe and through to the inhalation aperture.

A method and device for vaporizing phyto material for frictionally engaging a water pipe having an input port and an inhalation aperture with a water pipe fluid pathway formed therebetween comprising: a electronic vaporization element (EVE) comprising: an elongated hollow member having a first end and a second end opposite the first end, a fluid pathway propagating from the first end to the second end thereof, the second end for coupling with the water pipe input port, and a heating element disposed proximate the first end and comprising a first electrical contact and a second electrical contact the heating element comprising a resistive heater disposed between the first and second electrical contacts, the resistive heater for heating the phyto material disposed onto a phyto material contact surface to a predetermined temperature for vaporizing of the phyto material for creating a vapor therefrom and upon inhalation from the inhalation aperture this vapor is mixed with ambient air and flows through the fluid pathway from the first end and propagates through the input port of the water pipe and through to the inhalation aperture; a removable electrical power source comprising a first housing for having an electrical power source contained therein, the first housing comprising a frictional engagement mechanism for frictionally engaging of the water pipe; and, a first control circuit disposed within the first housing and electrically coupled with the electrical power source and the first and second electrical contacts of the EVE with an electronic vaporization element coupling cable electrically disposed therebetween, the first control circuit for providing electrical power from the electrical power source to the heating element for heating the phyto material disposed onto the phyto material contact surface to the predetermined temperature.

### BRIEF DESCRIPTION OF THE SEVERAL VIEWS OF THE DRAWINGS

FIG. 1A illustrates a electronic vaporization element in the form of a first electronic vaporization element;

FIG. 1B illustrates a fluid pathway formed in the first electronic vaporization element;

FIG. 1C illustrates a top view of the first electronic vaporization element;

FIG. 1D illustrates a bottom view of an annular heating element as part of the first electronic vaporization element;

FIG. 1E illustrates a perspective view of a electronic vaporization element in the form of a second electronic vaporization element;

FIG. 1F illustrates a cutaway view of a electronic vaporization element in the form of a second electronic vaporization element;

FIG. 1G illustrates a perspective view of a electronic vaporization element in the form of a third electronic vaporization element having a partial annular heating element;

FIG. 1H illustrates a bottom view of a electronic vaporization element in the form of a third electronic vaporization element having a partial annular heating element;

FIG. 1I illustrates a perspective view of a variation of the third electronic vaporization element having a partial annular heating element and a curved fluid pathway;

FIG. 2A illustrates a perspective view of device for vaporization of phyto material extracts coupled with a water pipe and in accordance with a first embodiment of the invention;

FIG. 2B illustrates a device for vaporization of phyto material extracts in accordance with the first embodiment of the invention from a top view;

FIG. 2C illustrates a device for vaporization of phyto material extracts in accordance with the first embodiment of the invention from an opened front view;

FIG. 2D illustrates a device for vaporization of phyto material extracts in accordance with the first embodiment of the invention from a side view;

FIG. 3A illustrates a device for vaporization of phyto material extracts in accordance with a second embodiment of the invention and attached to a water pipe;

FIG. 3B illustrates a device for vaporization of phyto material extracts in accordance with the second embodiment of the invention and showing an adjustable clamping mechanism;

FIG. 3B illustrates a device for vaporization of phyto material extracts in accordance with the second embodiment of the invention and showing a lead screw for adjusting of the adjustable clamping mechanism;

FIG. 3D illustrates a device for vaporization of phyto material extracts in accordance with the second embodiment of the invention and showing a control panel in a first position;

FIG. 3E illustrates a device for vaporization of phyto material extracts in accordance with the second embodiment of the invention and showing a control panel in a second position;

FIG. 3F illustrates a device for vaporization of phyto material extracts in accordance with the second embodiment of the invention and showing the adjustable clamping mechanism being frictionally engaged to a first diameter base water pipe;

FIG. 3G illustrates a device for vaporization of phyto material extracts in accordance with the second embodiment of the invention and showing the adjustable clamping mechanism being frictionally engaged to a second diameter base water pipe;

FIG. 3H illustrates a device for vaporization of phyto material extracts in accordance with the second embodiment of the invention and showing a plurality of batteries contained therein;

FIG. 3I illustrates a device for vaporization of phyto material extracts in accordance with the second embodiment of the invention and showing various input and output ports; and,

FIG. 3J a device for vaporization of

phyto material extracts in accordance with the second embodiment of the invention having a first magnet and a second magnet as part of the a second coupling port.

FIG. 4A shows a electronic vaporization element in accordance with a fifth embodiment of the invention and a device for vaporization of phyto materials in accordance with a third embodiment if the invention from a side view;

FIG. 4B shows a electronic vaporization element in accordance with a fifth embodiment of the invention and a device for vaporization of phyto material in accordance with a third embodiment if the invention from a top view;

FIG. 4C illustrates the electronic vaporization element in more details with a heating element shown being uncoupled from an elongated hollow member for clarity;

FIG. 4D illustrates a releasable locking mechanism from a side view where a separation between the first jaw and the second jaw is decreased for frictionally engaging of a water pipe;

FIG. 4E illustrates a releasable locking mechanism from a top view where a separation between the first jaw and the second jaw is decreased for frictionally engaging of a water pipe;

FIG. 4F illustrates a frictional engagement mechanism having a first jaw and a second jaw mechanically coupled to a lead screw from a side view and in a locked state;

FIG. 4G illustrates a frictional engagement mechanism having a first jaw and a second jaw mechanically coupled to a lead screw from a top view and in an unlocked state;

FIG. 5A illustrates a twist lock coupling for engaging of a water pipe with a first housing from a perspective view;

FIG. 5B illustrates a twist lock coupling from a top view with a water pipe a removed for clarity;

FIG. 5C illustrates a twist lock coupling in a locked mode of operation from a perspective view;

FIG. 5D illustrates a twist lock coupling in a locked mode of operation from a top view with a water pipe removed for clarity;

FIG. 6A illustrates a second control circuit disposed as part of the electronic vaporization element and in accordance with a seventh embodiment of the invention;

FIG. 6B illustrates an electronic vaporization element and with a first control circuit having a first lookup table 113a;

FIG. 6C illustrates a phyto material contact surface disposed between a resistive heater and a phyto material extract;

FIG. 6D illustrates a frictional engagement mechanism in the form of a suction cup;

FIG. 6E illustrates a voice recognition processor one of electrically and wirelessly coupled with a first control circuit;

FIG. 6F illustrates a cavity formed within a first housing for receiving of a voice recognition processor therein;

FIG. 6G illustrates a first control circuit coupled with at least a WIFI module and a Bluetooth® module for communicating with at least a smartphone and the internet;

FIG. 6H illustrates a tilt sensor electrically coupled with a first control circuit for determining whether a first housing has become knocked over;

FIG. 6I illustrates an adhesive tape envisaged for adhering of a water pipe to a rotating portion;

FIG. 6J illustrates an electronic vaporization element in accordance with an eight embodiment of the invention and having a pancake coil heater as a heating element;

FIG. 6K illustrates an electronic vaporization element in accordance with an eight embodiment and a pancake coil heater in more detail;

FIG. 6L illustrates a ninth embodiment of the invention with first and second power rails and first and second power couplings;

FIG. 7A illustrates a heating element is shown as a tubular heating element with a first temperature sensor a disposed inside of the tubular heating element;

FIG. 7B illustrates a resistive heater wrapped about a ceramic tube for forming a tubular heating element;

FIG. 7C illustrates an electronic vaporization element in accordance with a tenth embodiment of the invention where the heating element is in the form of a ceramic cup heating element and in a cutaway view;

FIG. 7D illustrates an electronic vaporization element in accordance with a tenth embodiment of the invention where the heating element is in the form of a ceramic cup heating element and in a perspective view;

FIG. 7E illustrates electronic vaporization element in the form of an eleventh embodiment of the invention as a removable cup electronic vaporization element in a cutaway view;

FIG. 7F a removable cup for an electronic vaporization element;

FIG. 7G illustrates a annular heater a from a top view as part of the eleventh embodiment of the invention;

FIG. 7H illustrate an electronic vaporization element in accordance with a twelfth embodiment of the invention where a heating element is in the form of a convection heating element; and

FIG. 8A illustrate robotic measured dose apparatus in accordance with a thirteenth embodiment of the invention.

### DETAILED DESCRIPTION OF EMBODIMENTS OF THE INVENTION

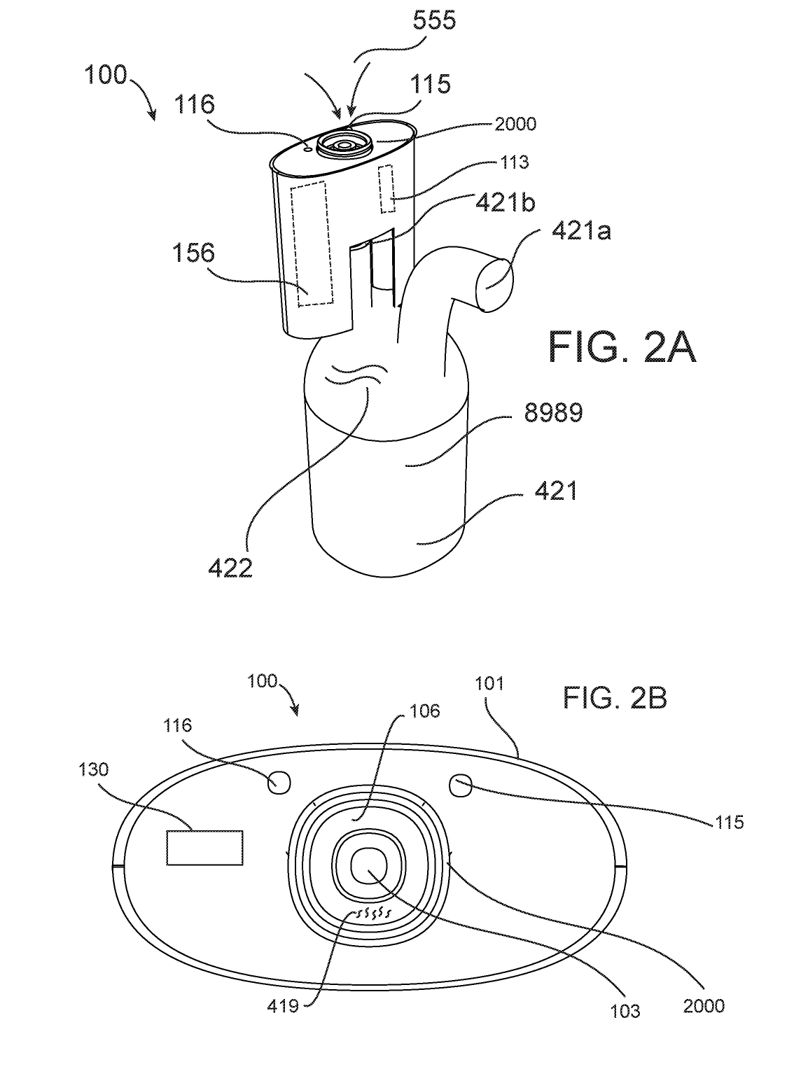

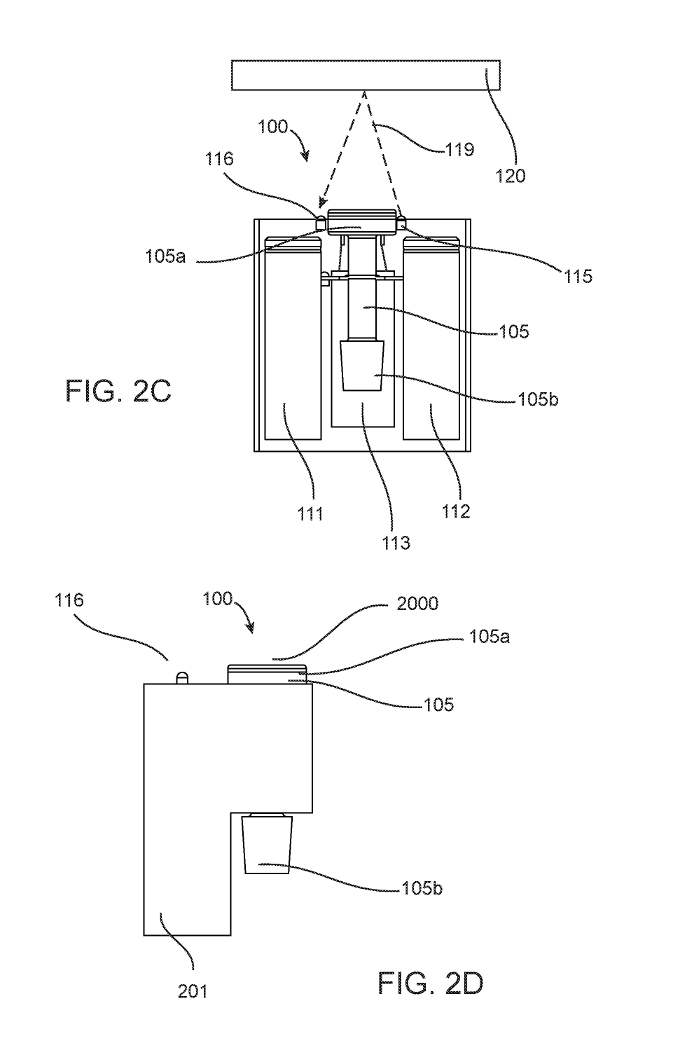

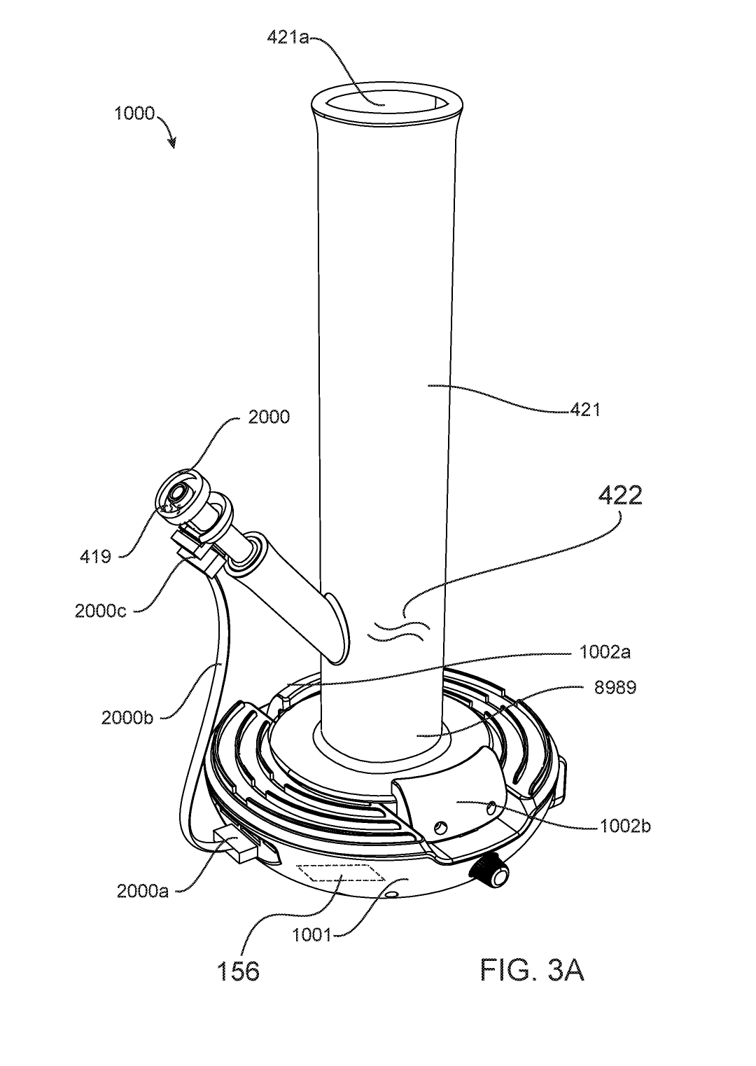

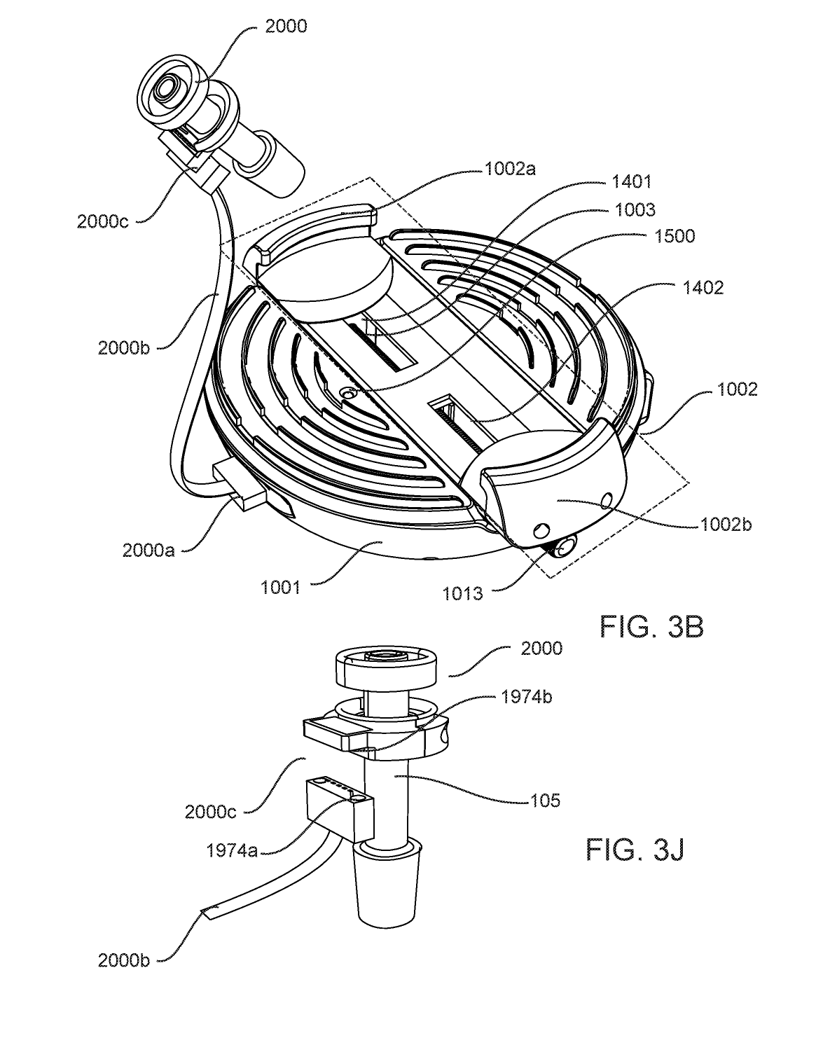

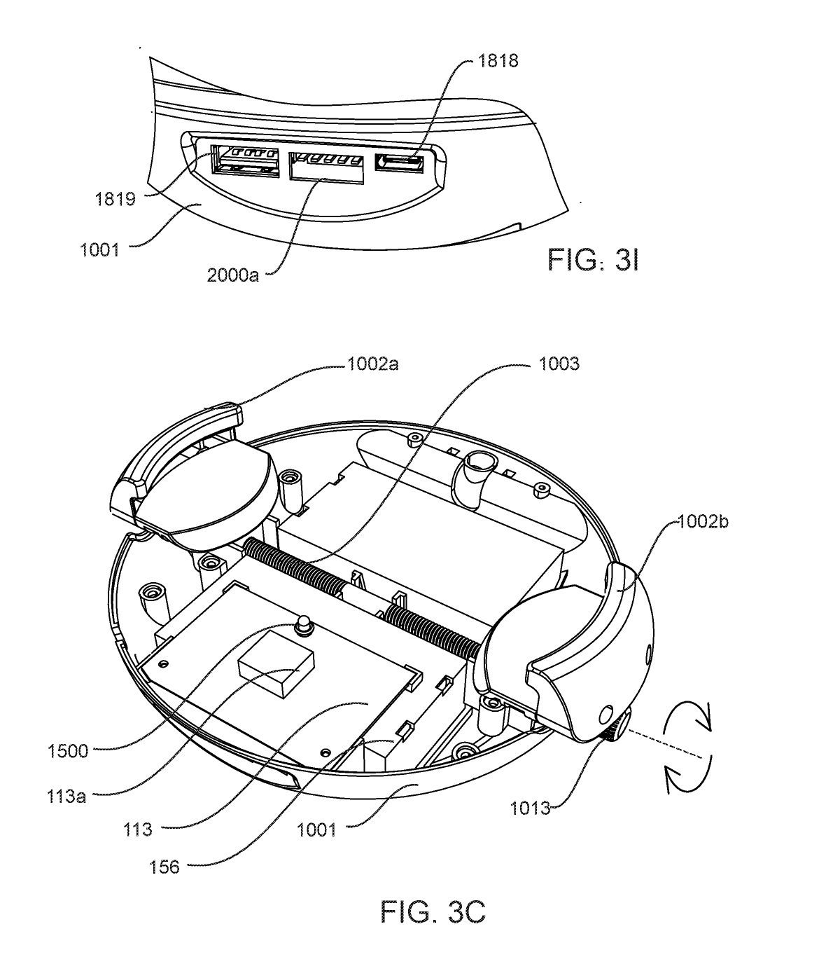

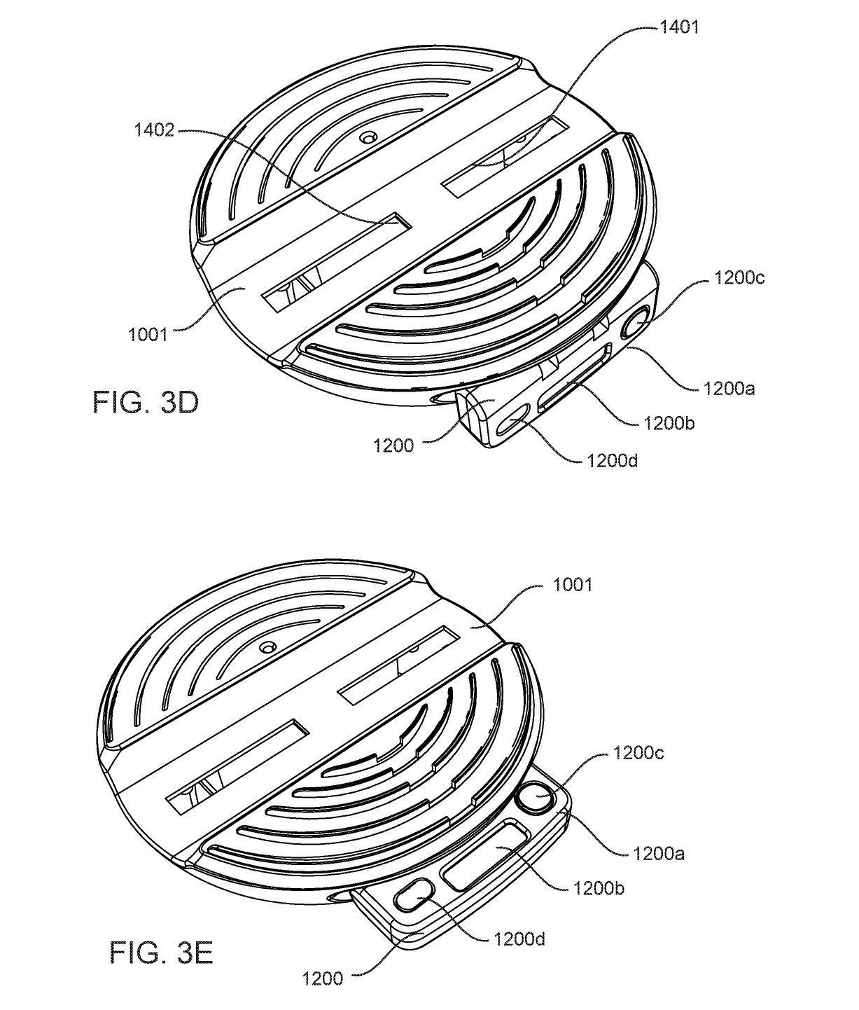

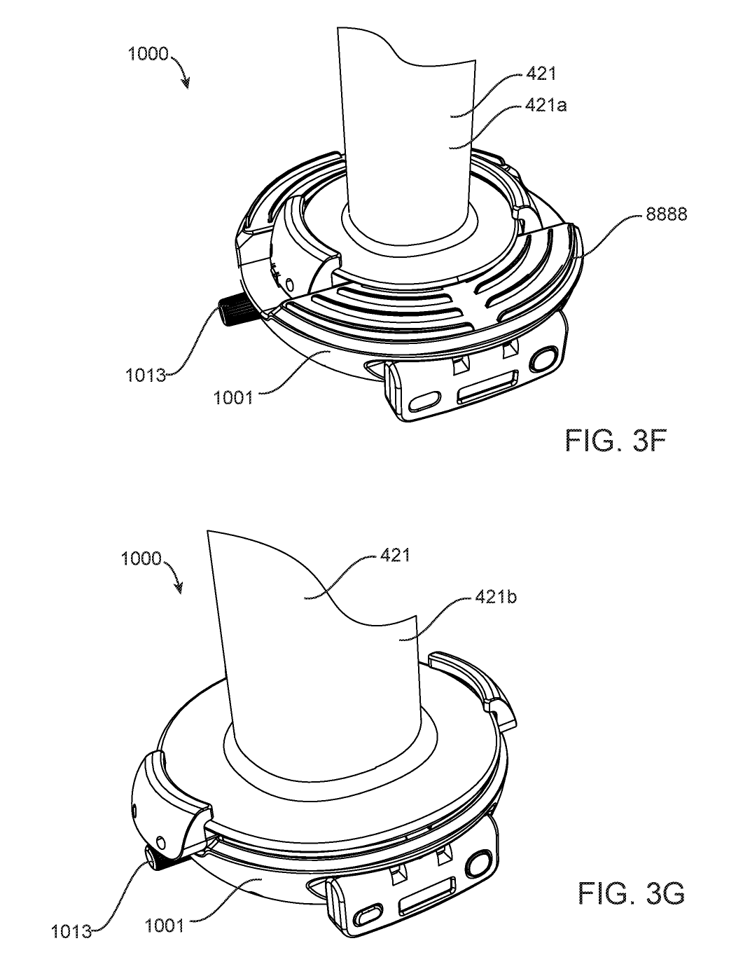

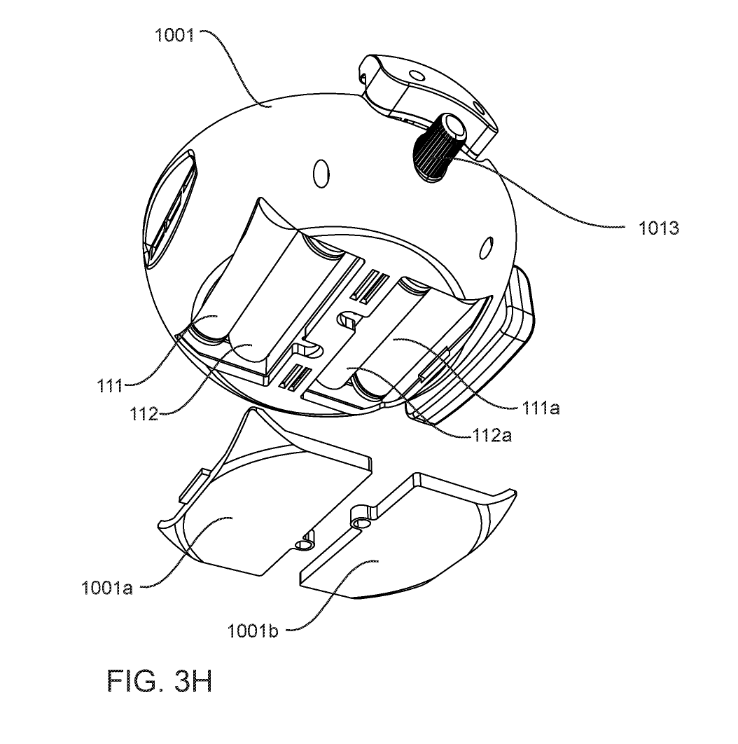

Phyto material extracts 419 are derived from phyto materials 420. Typically phyto materials 420 are leafy and phyto material extracts are oily or waxy in consistency. FIG. 2A illustrates a device for vaporization of phyto material extracts 100 (DVCPM) in accordance with a first embodiment of the invention. The DVCPM 100 is for attaching to a water pipe 421 having an input port 421b and an inhalation aperture 421a with a water pipe fluid pathway 8989 formed therebetween. FIG. 3A illustrates a device for vaporization of phyto material extracts 1000 (DVCPM) in accordance with a second embodiment of the invention. The DVCPM 1000 is for attaching to a water pipe 421 having an input port 421b and an inhalation aperture 421a with the water pipe fluid pathway 8989 formed therebetween.

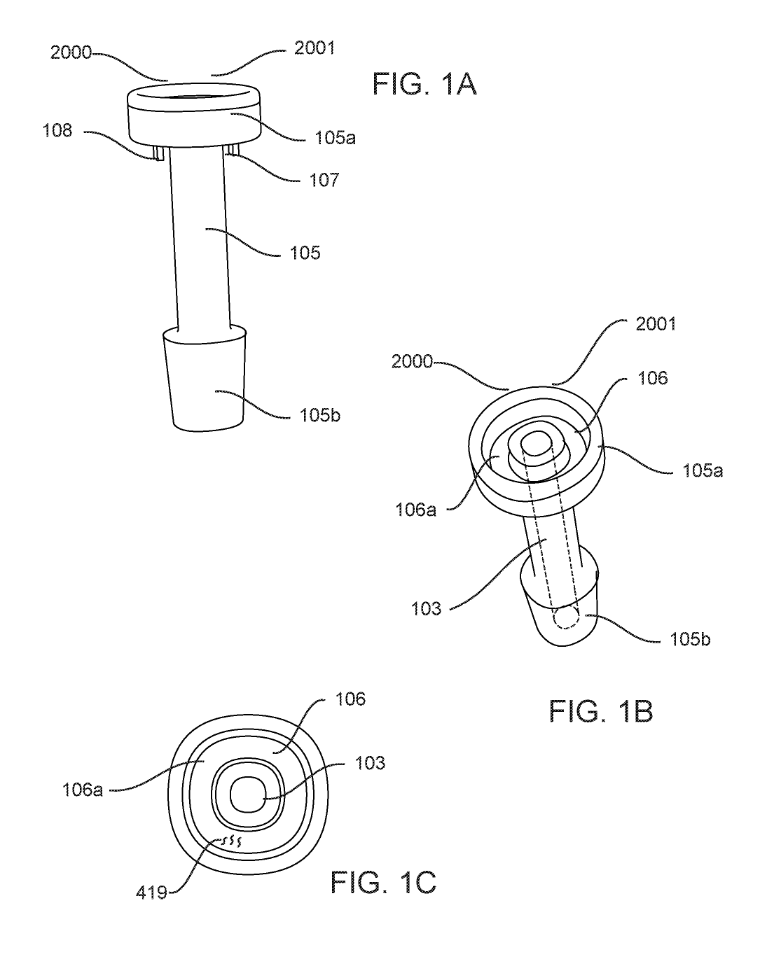

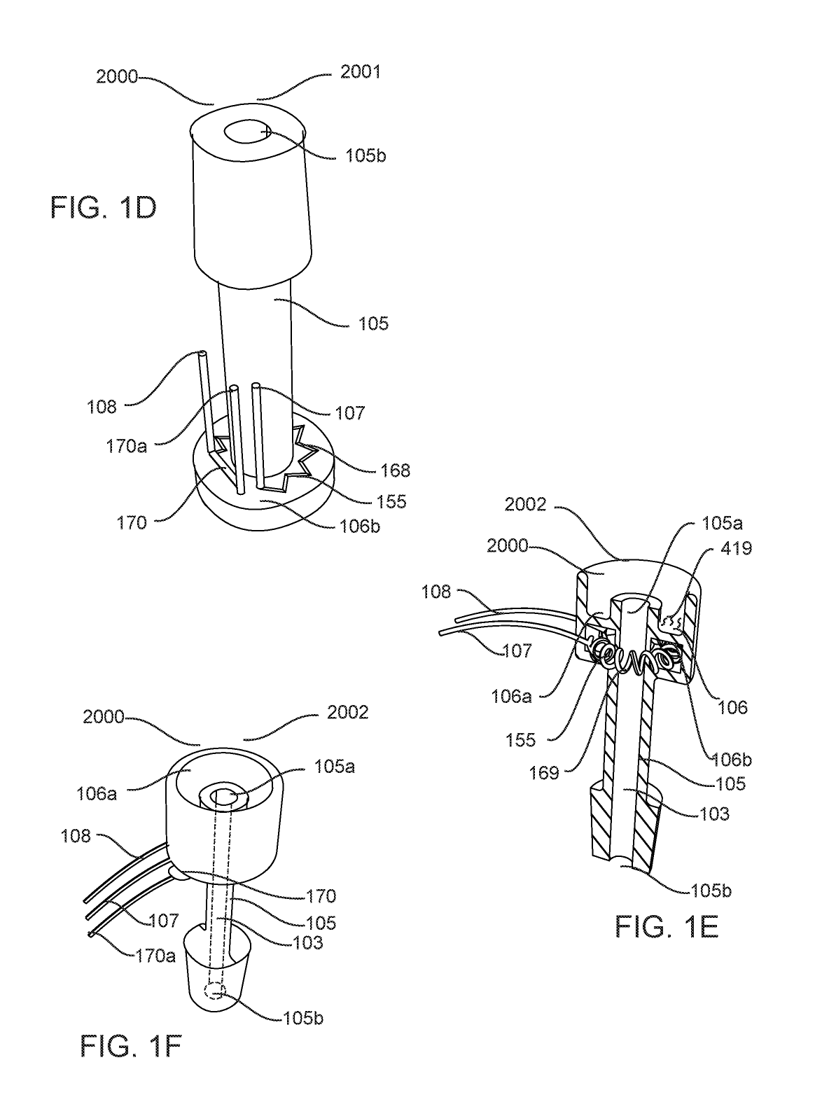

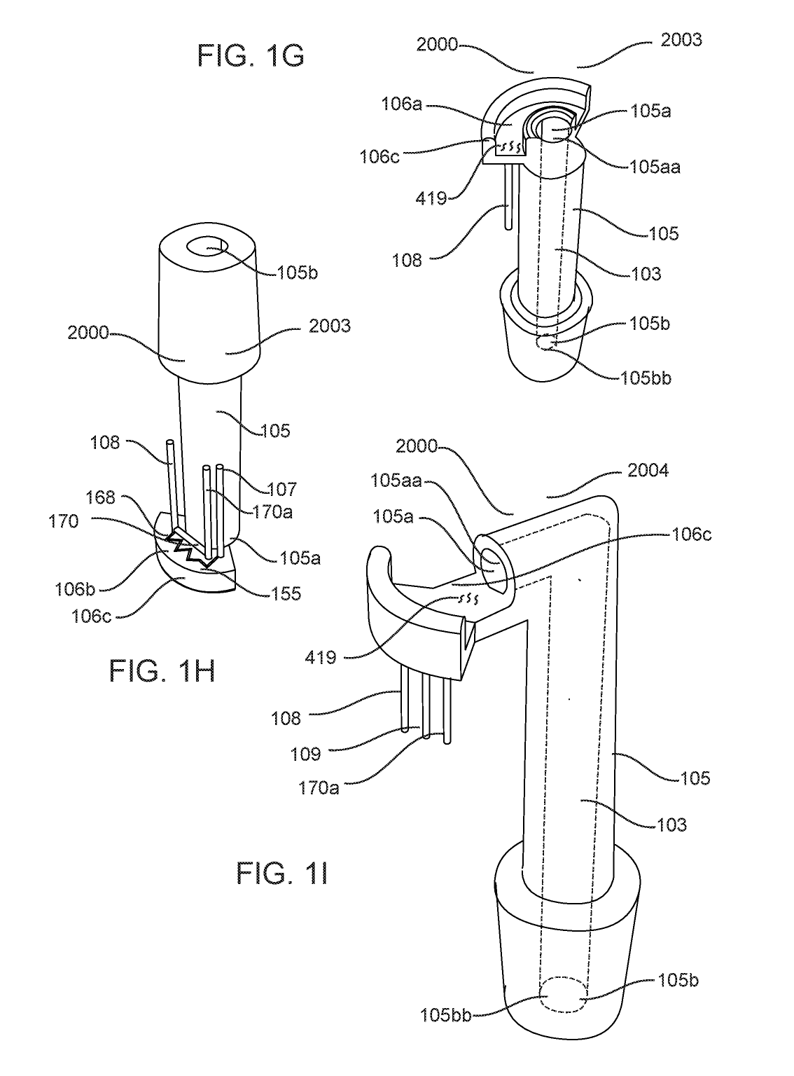

Referring to FIGS. 1A, 1B, 1C, 1D an electronic vaporization element 2000 is shown in the form of a first electronic vaporization element 2001. FIGS. 1E and 1F illustrate a electronic vaporization element 2000 in the form of a second electronic vaporization element 2002 and FIGS. 1G and 1H illustrates a electronic vaporization element 2000 in the form of a third electronic vaporization element 2003. FIG. 1I illustrates an electronic vaporization element 2000 in the form of a fourth electronic vaporization element 2004 that is a variation of the third electronic vaporization element 2003. Throughout the detailed description, the electronic vaporization element 2000 is for use in both of the first and second embodiments of the invention, DVCPM 100 and DVCPM 1000, respectively.

Referring to FIG. 1A, the electronic vaporization element 2000, in the form of a first electronic vaporization element 2001, is shown in perspective view and is formed from an elongated hollow member 105 that is made from a low thermal conductivity material, such as ceramic, and having a first end 105a and a second end 105b opposite the first end 105, a fluid pathway 103 (as seen in FIG. 1B) propagates through the elongated hollow member 105 from the first end 105a to the second end 105b thereof. The second end 105b is for coupling with the water pipe input port 421b, as shown in FIGS. 2A and 3A.

The electronic vaporization element 2000 has an annular heating element 106 having a first side 106a and a second side 106b (FIG. 1D) opposite the first side 106a, the annular heating element 106 is thermally coupled with the elongated hollow member 105 proximate the first end 105a having the first side 106a facing the first end 105a with the fluid pathway 103 propagating through a center thereof (as seen in FIG. 1B), the annular heating element 106 comprises a first electrical contact 107 and a second electrical contact 108 proximate the second side 106b. The annular heating element 106 secured to the elongated hollow member 105 for allowing thermal expansion thereof along a radial axis perpendicular to the fluid pathway 103. Without properly securing the annular heating element 106 to the elongated hollow member 105 it is easy to crack the annular heating element 106 due to expansion forces of the elongated hollow member 105 and as such a unitary construction of the annular heating element 106 is preferable.

Referring to FIG. 1D, the annular heating element 106 comprising a resistive heater 155 disposed between the first and second electrical contacts, 107 and 108, and proximate the second side 106b. The annular heating element 106 comprises ceramic material where the resistive heater 155 comprises a metallic planar heater 168 disposed on the second side 106b between the first and second electrical contacts 107 108 for receiving of electrical energy from the electrical power source 156, wherein the thermal coupling between the annular heating element and the elongated hollow member 105 comprises silica material. Silica is also known in the art as ceramic glaze, so the coupling between the annular heating element 106 and the elongated hollow member 105 is by means of a ceramic glaze. Another type of ceramic glaze know in the art is manufactured from aluminum oxide.

The electrical power source 156 is electrically coupled with the first and second electrical contacts 107 108 for providing of electrical power to the resistive heater 155 for heating of the resistive heater 155 for imparting thermal energy to the annular heating element 106.

As is evident from FIG. 1D, the electronic vaporization element 2000 comprises a temperature sensor 170 thermally coupled with at least one of the elongated hollow member 105 and the annular heating element 106 proximate the second side 106b of the annular heating element 106, the temperature sensor 170 has a temperature signal output port 170a for providing a temperature signal in dependence upon the imparting of thermal energy to the annular heating element 106. Typically the temperature signal is based on a resistance of the temperature sensor 170 and the resistance varies inversely with respect to the temperature being sensed by the temperature sensor 170. Optionally, the temperature sensor is in the form of a thermocouple as is known in the art of measuring temperatures.

Referring to FIG. 2A, the DVCPM 100 in accordance with the first embodiment of the invention is shown attached to a water pipe 421 having an inhalation aperture 421a and an input port 421b. The electronic vaporization element 2000, for example the first electronic vaporization element 2001, but it is not limited to the first electronic vaporization element 2001, the second electronic vaporization element 2002 or the third electronic vaporization element 2003 or the fourth electronic vaporization element 2004, any of the electronic vaporization elements 2000 are useable with the DVCPM 100.

In this embodiment the electronic vaporization element 2000 is disposed within the first housing 101 and the first housing 101 frictionally engages the elongated hollow member 105 where the second end 105b of the elongated hollow member 105 couples with the water pipe input port 421b. An electrical power source 156 (disposed within the first housing 101 and not visible from an outside thereof, but visible in FIG. 2C as the first and second batteries 111, 112) is provided and coupled with a first control circuit 113 electrically coupled with the electrical power source 156 (FIG. 2C) and the first and second electrical contacts 107 108 and the temperature signal output port 170a. The first control circuit 113 for processing of the temperature signal and for controllably providing of the electrical power to the resistive heater 155 for reaching the predetermined temperature of the second side 106b of the annular heating element.

During heating of the resistive heater 155, a portion of the thermal energy is transferred to the annular heating element 106 first side 106a and another portion, other than the first portion, is transferred to the elongated hollow member 105 proximate the first end 105a, upon the annular heating element 106 second side 106b, as the phyto material contact surface, reaching a predetermined temperature the phyto material extract 419 is applied to the annular heating element 106 first side 106b (FIG. 1C) and becomes vaporized and upon inhalation from the inhalation aperture 421a this vapor 422 is mixed with ambient air 555 (FIG. 2A) and flows through the fluid pathway 103 from the first end 105a where it receives thermal energy proximate the coupling between the annular heating element 106 and the elongated hollow member 105 and loses thermal energy to the elongated hollow member 105 proximate the second end 105b as it propagates through the input port 421b of the water pipe 421 and through to the inhalation aperture 421a.

Referring to FIGS. 1E and 1F, the electronic vaporization element 2000, in the form of the second electronic vaporization element 2002, is shown in perspective view and cutaway view, respectively, and is formed from an elongated hollow member 105 that is made from a low thermal conductivity material, such as glass or quartz, and having a first end 105a and a second end 105b opposite the first end 105, a fluid pathway 103 (as seen in FIG. 1F) propagates through the elongated hollow member 105 from the first end 105a to the second end 105b thereof. The second end 105b is for coupling with the water pipe input port 421b, as shown in FIGS. 2A and 3A.

The electronic vaporization element 2000 has an annular heating element 106 having a first side 106a and a second side 106b opposite the first side 106a, the annular heating element 106 is thermally coupled with the elongated hollow member 105 proximate the first end 105a having the first side 106a facing the first end 105a with the fluid pathway 103 propagating through a center thereof (as seen in FIG. 1F), the annular heating element 106 comprising a first electrical contact 107 and a second electrical contact 108 proximate the second side 106b, the annular heating element 106 secured to the elongated hollow member 105 for allowing thermal expansion thereof along a radial axis perpendicular to the fluid pathway 103.

Referring to FIG. 1E, a cutaway view of the electronic vaporization element 2000, in the form of the second electronic vaporization element 2002, is shown. The annular heating element 106 comprising a resistive heater 155 disposed between the first and second electrical contacts, 107 and 108, and proximate the second side 106b. The resistive heater 155 comprises a resistance wire 169 disposed proximate the second side 106b between the first and second electrical contacts 107 108 for receiving of electrical energy from the electrical power source 156, wherein the thermal coupling between the annular heating element and the elongated hollow member 105 comprises glass or quartz.

The electrical power source 156 is electrically coupled with the first and second electrical contacts 107, 108 for providing of electrical power to the resistive heater 155 for heating of the resistive heater 155 for imparting thermal energy to the annular heating element 106.

Referring to FIG. 2A for example, when the second electronic vaporization element 2002 is utilized and during heating of the resistive heater 155, a portion of the thermal energy is transferred to the annular heating element 106 first side 106a and another portion, other than the first portion, is transferred to the elongated hollow member 105 proximate the first end 105a, upon the annular heating element 106 second side 106b reaching the predetermined temperature the phyto material extract 419 is applied to the annular heating element 106 first side 106b (FIG. 1E) and becomes vaporized and upon inhalation from the inhalation aperture 421a this vapor 422 is mixed with ambient air 555 and flows through the fluid pathway 103 from the first end 105a where it receives thermal energy proximate the coupling between the annular heating element 106 and the elongated hollow member 105 and loses thermal energy to the elongated hollow member 105 proximate the second end 105b as it propagates through the input port 421b of the water pipe 421 and through to the inhalation aperture 421a.

Referring to FIG. 1F, the electronic vaporization element 2000 comprises a temperature sensor 170 thermally coupled with at least one of the elongated hollow member 105 and the annular heating element 106 proximate the second side 106b of the annular heating element 106, the temperature sensor 170 has a temperature signal output port 170a for providing a temperature signal in dependence upon the imparting of thermal energy to the annular heating element 106. In some cases uses a glass or quartz electronic vaporization element 2000 is preferable because a user can see the resistance wire 169 heating up and it provides a glow as the predetermined temperature is reached.

Referring to FIGS. 1G and 1H, the electronic vaporization element 2000 is shown in the form of the third electronic vaporization element 2003. The electronic vaporization element 2000 in the form of the third electronic vaporization element 2003 is formed from an elongated hollow member 105 that is made from a low thermal conductivity material, such as ceramic, but can also be made from glass or quartz, and having a first end 105a and a second end 105b opposite the first end 105, the fluid pathway 103 (as seen in FIG. 1G) propagates through the elongated hollow member 105 from the first end 105a to the second end 105b thereof. The second end 105b is for coupling with the water pipe input port 421b, as shown in FIGS. 2A and 3A.

The electronic vaporization element 2000 has a annular heating element 106 that is a partial annular heating element 106c that does not comprise a full three hundred and sixty degrees arc about the fluid pathway 103 when thermally coupled about the elongated hollow member 105 and has a portion thereof removed, wherein it comprise about a ninety degrees arc about the fluid pathway when disposed about the elongated hollow member 105.

The partial annular heating element 106c is radially disposed with respect to the elongated hollow member 105. As shown in FIG. 1G, the elongated hollow member 105 comprises a first aperture 105aa proximate the first end thereof 105a and a second aperture 105bb proximate the second end thereof 105a and the fluid pathway 103 formed between the first and second apertures, 105aa and 105bb, wherein the first and second apertures are axially disposed and comprises the resistive heater 155. Preferably the partial annular heating element 106c is disposed proximate the first end 105a of the elongated hollow member 105.

The partial annular heating element 106c has a first side 106a and a second side 106b opposite the first side 106a, partial annular heating element 106c is thermally coupled with the elongated hollow member 105 proximate the first end 105a having the first side 106a facing the first end 105a with the fluid pathway 103 propagating through a center thereof (as seen in FIG. 1G), the partial annular heating element 106c comprising a first electrical contact 107 and a second electrical contact 108 proximate the second side 106b, the partial annular heating element 106c secured to the elongated hollow member 105 for allowing thermal expansion thereof along a radial axis that is perpendicular to the fluid pathway 103.

Referring to FIG. 1H, the partial annular heating element 106c comprising a resistive heater 155 disposed between the first and second electrical contacts, 107 and 108, and proximate the second side 106b. The partial annular heating element 106c comprises ceramic material where the resistive heater 155 comprises a metallic planar heater 168 disposed on the second side 106b between the first and second electrical contacts 107 108 for receiving of electrical energy from the electrical power source 156, wherein the thermal coupling between the partial annular heating element 106c and the elongated hollow member 105 comprises silica material.

The electrical power source 156 is electrically coupled with the first and second electrical contacts 107 108 for providing of electrical power to the resistive heater 155 for heating of the resistive heater 155 for imparting thermal energy to the partial annular heating element 106c.

Referring to FIG. 2A, when the electronic vaporization element 2000 in the form of the third electronic vaporization element 2003 is coupled with the water pipe 421, during heating of the resistive heater 155, a portion of the thermal energy is transferred to the partial annular heating element 106c first side 106a and another portion, other than the first portion, is transferred to the elongated hollow member 105 proximate the first end 105a, upon the partial annular heating element 106c second side 106b reaching the predetermined temperature the phyto material extract 419 is applied to the partial annular heating element 106c first side 106b (FIG. 1G) and becomes vaporized and upon inhalation from the inhalation aperture 421a this vapor 422 is mixed with ambient air 555 and flows through the fluid pathway 103 from the first end 105a where it receives thermal energy proximate the coupling between the partial annular heating element 106c and the elongated hollow member 105 and loses thermal energy to the elongated hollow member 105 proximate the second end 105b as it propagates through the input port 421b of the water pipe 421 and through to the inhalation aperture 421a.

Referring to FIG. 1H, the electronic vaporization element 2000 comprises a temperature sensor 170 thermally coupled with at least one of the elongated hollow member 105 and the partial annular heating element 106c proximate the second side 106b of the partial annular heating element 106c, the temperature sensor 170 has a temperature signal output port 170a for providing a temperature signal in dependence upon the imparting of thermal energy to the partial annular heating element 106c.

FIG. 1I illustrates a variation of the third electronic vaporization element 2003 having the partial annular heating element 2003 in the form of a fourth electronic vaporization element 2004, whereby the resistive heater 155 (not visible in this FIG. 1I) is disposed between the first and second electrical contacts, 107 and 108, is at a distance, for example 20 mm, from an axial center of the second end 105a of the elongated hollow member 105. Whereby in comparison, for the third electronic vaporization element 2003 the resistive heater 155 is approximately 6 mm away from the axial center of the second end 105a of the elongated hollow member 105.

Furthermore, the fluid pathway 103 is curved between the first end 105a and the second end 105b. Such a variation may be preferable so that thermal transfer from the fourth electronic vaporization element 2004 to the hollow ceramic member 105 is reduced as well the fourth electronic vaporization element 2004 provides for a lower thermal inertia than the first electronic vaporization element 2001.

The elongated hollow member 105 comprises a first aperture 105aa proximate the first end thereof 105a and a second aperture 105bb proximate the second end thereof 105a and the fluid pathway 103 formed between the first and second apertures, wherein the first and second apertures 105aa and 105bb are other than axially disposed and preferably central axes of the first and second apertures 105aa and 105bb are perpendicular to each other.

In this fourth electronic vaporization element 2004 the resistive heater 155 is radially disposed away from the elongated hollow member 105, which therefore results in a bend in the fluid pathway 103. Using the fourth electronic vaporization element 2004 is sometimes preferable as it allows for an elongated path length for the fluid pathway 103 and as such improved cooling for the vapor 422 as it propagates through the fluid pathway 103. If the fourth electronic vaporization element 2004 uses quartz material then the resistive heater 155 is envisaged comprising a pancake ceramic heater or a resistance wire 169. If the fourth electronic vaporization element 2004 uses a ceramic material then the resistive heater 155 is envisaged comprising a metallic planar heater 168 that is sintered onto the ceramic.

Referring to FIG. 2A and in conjunction with FIGS. 2A, 2B and 2D a first infrared transmitter 115 is envisaged for protruding past the first housing 101 proximate the first end 105a of the electronic vaporization element 2000. FIG. 2B illustrates a top view and FIG. 2C illustrates an internal front view and FIG. 2D illustrates a closed side view.

A first infrared receiver 116 is provided for protruding past the first housing 101 proximate the first end 105a of the electronic vaporization element 2000, the first infrared transmitter 115 and the first infrared receiver 116 are electrically coupled with the first control circuit 113, the first infrared transmitter 115 for sending out a first infrared signal 119 for being reflected from an infrared signal reflective member 120 for being received by the first infrared receiver 116 for enabling the heating of the annular ceramic heating element 106 and for other than being received by the first infrared receiver 116 when the infrared signal reflective member 120 is other than present, upon heating of the annular ceramic heating element 106, the phyto material extract 419 is heated to the predetermined temperature and becomes vaporized and this vapor 422 and is mixed with ambient air 555 and flows through the fluid pathway 103, as illustrated in FIG. 2A.

Preferably the infrared signal reflective member 120 is in the form of a hand, whereby when the hand of a user is waived over the top of the DVCPM 100, this activates the first control circuit 113 for heating of the electronic vaporization element 2000. Referring to FIG. 2C, a first battery 111 and a second battery 112 are shown as part of the electrical power source 156. Any of the electronic vaporization elements 2000 in the form of the first through fourth, 2001 through 2004, are envisaged to work with the first infrared transmitter 115 and the first infrared receiver 116.

FIG. 3A illustrates a device for vaporization of phyto material extracts 1000 (DVCPM) in accordance with a second embodiment of the invention. The DVCPM 1000 is for attaching to a water pipe 421 having an input port 421b and an inhalation aperture 421a with a water pipe fluid pathway 8989 formed therebetween. The DVCPM 1000 includes the electronic vaporization element 2000 having the second end 105b coupled with the input port 421b. The water pipe 421 has a first housing 1001 for preferably having the an electrical power source 156 contained therein, the first housing 1001 comprising an adjustable clamping mechanism 1002, as is shown in FIG. 3B, for frictionally engaging of the water pipe 421.

FIG. 3A furthermore illustrates a electronic vaporization element first coupling port 2000a electrically coupled with the first control circuit 113 (FIG. 3C) and electronic vaporization element second coupling port 2000c electrically coupled with the electronic vaporization element 2000 first and second electrical contacts 107 108 and the temperature signal output port 170a.

A electronic vaporization element connector cable 2000b is electrically coupled between the electronic vaporization element first coupling port 2000a and the electronic vaporization element second coupling port 2000c, the electronic vaporization element connector cable 2000b is for electrically coupling of the electronic vaporization element 2000 with the first control circuit 113 (FIG. 3C).

Preferably the electronic vaporization element connector cable 2000b is magnetically and electrically coupled with the electronic vaporization element whereby the second coupling port 2000c comprises a magnetic coupling. FIG. 3j illustrates a first magnet 1974a and a second magnet 1974b whereby the second coupling port 2000c is electrically and mechanically held together using the first and second magnets 1974a and 1974b. However a standard pin connector is also envisaged as would be obvious to one skilled in the art.

Referring to FIG. 3B, the adjustable clamping mechanism 1002 comprises a first jaw 1002a and a second jaw 1002b disposed opposite the first jaw 1002a, the first and second jaws are mechanically coupled to a lead screw 1003, for upon rotating of the lead screw 1003 in a clockwise direction for increasing a frictional engagement of the water pipe 421 and for upon rotating of the lead screw in a counter clockwise direction for decreasing a frictional engagement of the water pipe 421, wherein a spacing between the first jaw 1002a and the second jaw 1002b varies between 6 cm and 15 cm, the first and second jaws 1002a and 1002b for respectively sliding within a first track 1401 and a second track 1402. A thumb screw 1013 is provided and frictionally coupled with the lead screw 1003 and at least partially protruding past the first housing 1001 for being turned to adjust the lead screw 1003.

This allows the end user the possibility to adjust the adjustable clamping mechanism 1002 to accommodate various water pipe bases. FIG. 3F illustrates the water pipe 421 as a first diameter base water pipe 421a being frictionally engaged by the adjustable clamping mechanism 1002 when the first and second jaws 1002a and 1002b are in a first position and FIG. 3G illustrates the water pipe 421 as a second diameter base water pipe 421b being frictionally engaged by the adjustable clamping mechanism 1002 when the first and second jaws 1002a and 1002b are in a second position. Because the second diameter base water pipe 421b is of a larger diameter than the first diameter base water pipe 421a, a spacing between the first and second jaws is larger in the second position than the first position. Additionally shown in FIG. 3F is a plurality of deformable ribs 8888 used for assisting in frictionally contacting the water pipe 421 when its frictionally engaged by the adjustable clamping mechanism 1002.

A three colored LED 1500 is also provided and protrudes past the first housing 1001 and is optically aimed at the water pipe 421. The LED 1500 electrically coupled with the first control circuit 113, the LED 1500 for directing light towards the water pipe 421 and for changing color in dependence upon the temperature signal. For example the LED 1500 has a blue color when a temperature of the resistive heater 155 is around 200 degrees Fahrenheit and has a red color when the temperature of the resistive heater 155 is around 600 degrees Fahrenheit.

Referring to FIG. 3C, the first control circuit 113 electrically coupled with the electrical power source 156 and the first and second electrical contacts 107 108 of the electronic vaporization element and the temperature signal output port 170a, the first control circuit 131 includes a first processor 113a for processing of the temperature signal and for controllably providing of the electrical power to the resistive heater 155 for reaching the predetermined temperature.

Referring to FIGS. 3D and 3E, a control panel 1200 is provided having a control surface 1200a, the control panel 1200 is rotationally coupled with the first housing 1001, the control panel being hinged with the first housing 1001 for operating between a first position (FIG. 3D) and a second position (FIG. 3E), wherein in the first position the control surface 1200a is approximately perpendicular to the first track 1401 and the second track 1402 and where in the second position the control surface 1200a is approximately parallel to the first track 1401 and the second track 1402.

Furthermore, the control panel 1200 comprises an OLED display screen 1200b electrically coupled with the first control circuit 113 for displaying a temperature in dependence upon the temperature control signal and an activation button 1200c electrically coupled with the first control circuit 113 for enabling operation of the first control circuit 113 and a temperature adjustment rocker button 1200d electrically coupled with the first control circuit 113 for adjusting the predetermined temperature from, for example 100 degrees Celsius to 400 degrees Celsius.

FIG. 3H illustrates the DVCPM 1000 from a bottom view of the first housing 1001 and showing a plurality of batteries 111, 112, 111a, 112a as the electrical power source 156, the plurality of batteries 111, 112, 111a, 112a electrically coupled in series and electrically coupled with the first control circuit 113, wherein the first housing 1001 comprises a first battery door 1001a and a second battery door 1001b, wherein the batteries 111 and 112 are removable through the first battery door 1001a and the batteries 111a and 112a are removable through the second battery door 1001b. The plurality of batteries are preferably 18650 lithium ion batteries.

FIG. 3I illustrates the DVCPM 1000 with various input and output ports, such as a USB-C port 1818 for receiving of electrical energy from a recharger (not shown) and a USB port 1819 for providing of electricity from the electrical power source 156 to connected external devices for being recharged, such as a cellular phone. The electronic vaporization element first coupling port 2000a is also oriented proximate the USB-C and the USB port and these ports are electrically coupled with the first control circuit 113. The DVCPM 1000 thus can also act as a portable battery bank for recharging other electrical devices and for storing electrical energy therein for portable heating of the electronic vaporization element 2000.

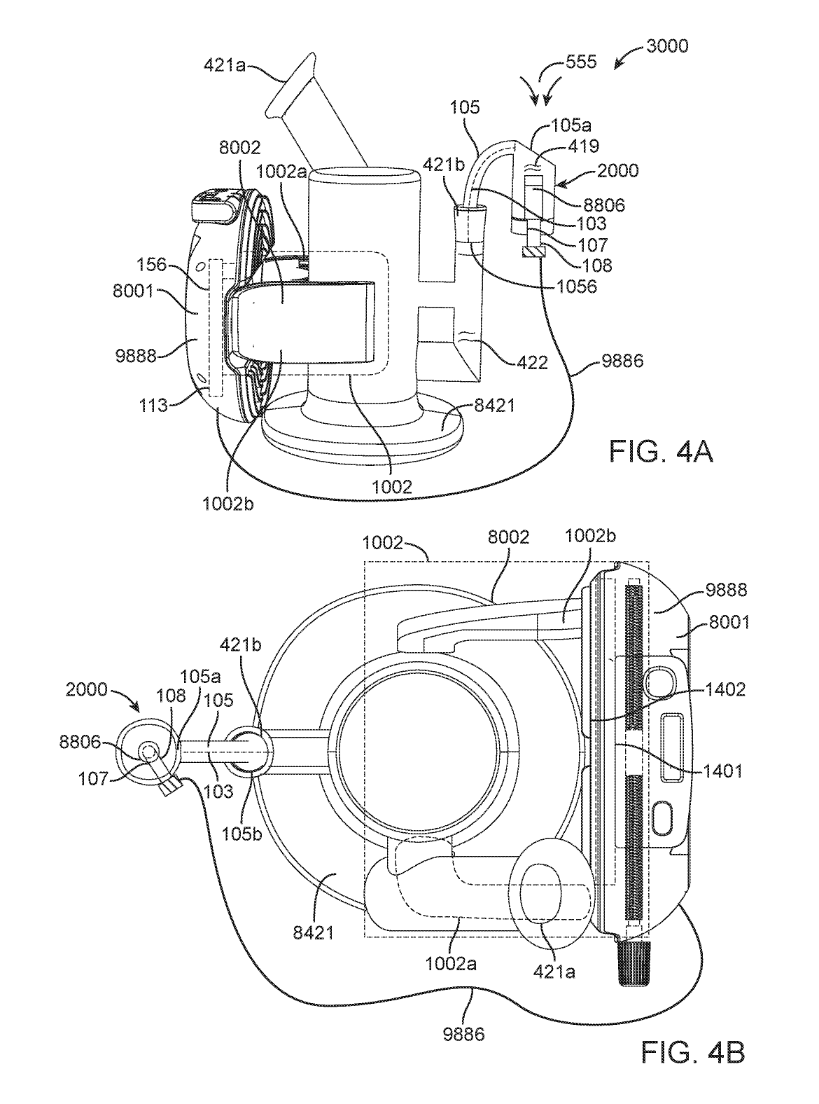

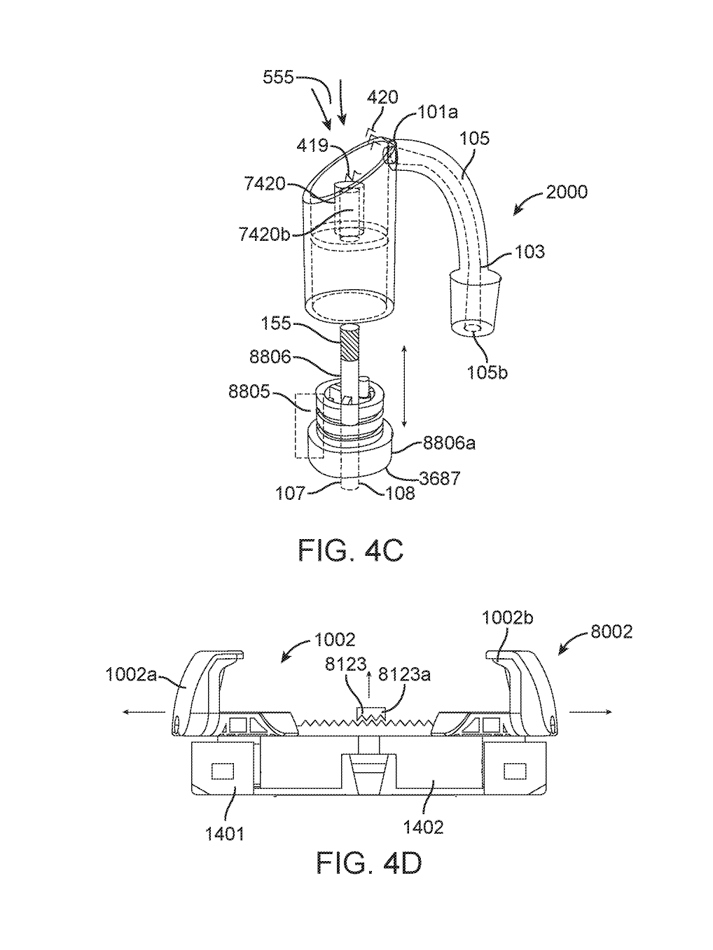

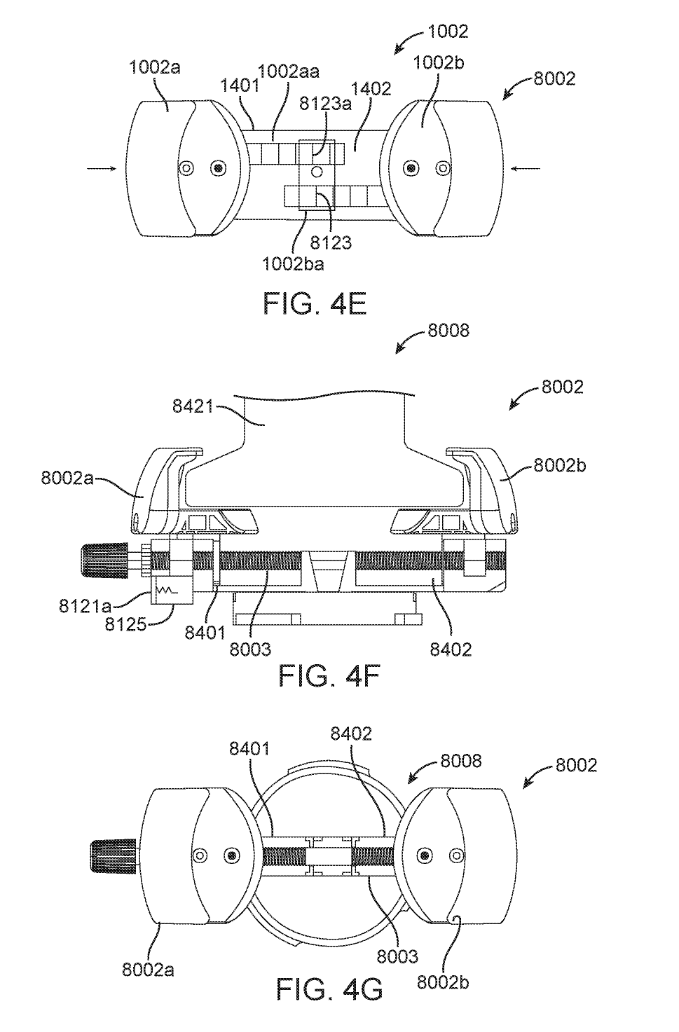

FIG. 4A illustrates a method and device for vaporizing phyto material is shown in accordance with a third embodiment of the invention 3000. This is shown in FIG. 4A from a side view and in FIG. 4B from a top view. A water pipe 8421 is shown being coupled with an electronic vaporization element 2000 (EVE) in accordance with fifth electronic vaporization element. The EVE 2000 is formed from an elongated hollow member 105 having a first end 105a and a second end 105b opposite the first end 105, a fluid pathway 103 propagating from the first end 105a to the second end 105b thereof, the second end 105b for coupling with the water pipe input port 421b.

Referring to FIG. 4C, the EVE 2000 is shown in more details with a heating element 8806 shown being uncoupled from the elongated hollow member 105 for clarity. The EVE 2000 is formed from an elongated hollow member 105 comprising a low thermally conductivity material, such as glass or ceramic, and having a first end 105a and a second end 105b opposite the first end 105, a fluid pathway 103 propagating from the first end 105a to the second end 105b thereof, the second end 105b for coupling with the water pipe input port 421b (FIG. 4A), the elongated hollow member 105 proximate the first end 105a having a phyto material contact surface 7420 and having disposed opposite thereof a second side phyto material contact surface 7420b, the phyto material extract 419 is for being applied to the phyto material contact surface 7420b proximate the first end 105a. The heating element 8806 comprising a first electrical contact 107 and a second electrical contact 108 and disposed proximate the first end 105a and in proximity of the second side phyto material contact surface 7420b and opposite the phyto material contact surface 7420, the heating element being partially disposed within a heating element housing 8806a, with the resistive heater 155 protruding past the heating element housing 8806a.

A frictional coupling 8805 is formed between the heating element housing 8806a and the elongated hollow member 105 proximate the first end 105a for releasably coupling of the heating element 8806 with the elongated hollow member 105 proximate the first end 105a. The heating element 8806 for applying heat to the second side phyto material contact surface 7420b and for a portion of the applied heat to radiate through the elongated hollow member 105 proximate the first end 105a into the to the phyto material contact surface 7420 to which the phyto material extract 419 is applied, the phyto material contact surface 7420 for heating of the phyto material extract 419 by the radiated portion of the applied heat to a predetermined temperature for vaporizing of the phyto material for creating a vapor 422 therefrom and upon inhalation from the inhalation aperture 421a this vapor 422 is mixed with ambient air 555 and flows through the fluid pathway 103 from the first end 105a and propagates through the input port 421b of the water pipe 421 and through to the inhalation aperture 421a.

As is shown in FIG. 4A, the removable electrical power source 9888 comprising the first housing 8001 for having an electrical power source 156 contained therein, the first housing 8001 comprising the frictional engagement mechanism 8002 for frictionally engaging of the water pipe 421 and the first control circuit 113 disposed within the first housing 8001 and electrically coupled with the electrical power source 156 and the first and second electrical contacts 107 108 of the EVE 2000 with the electronic vaporization element coupling cable 9886 electrically disposed therebetween, the first control circuit 113 for providing electrical power from the electrical power source 156 to the heating element 8806 for heating the phyto material extract 419 to the predetermined temperature.

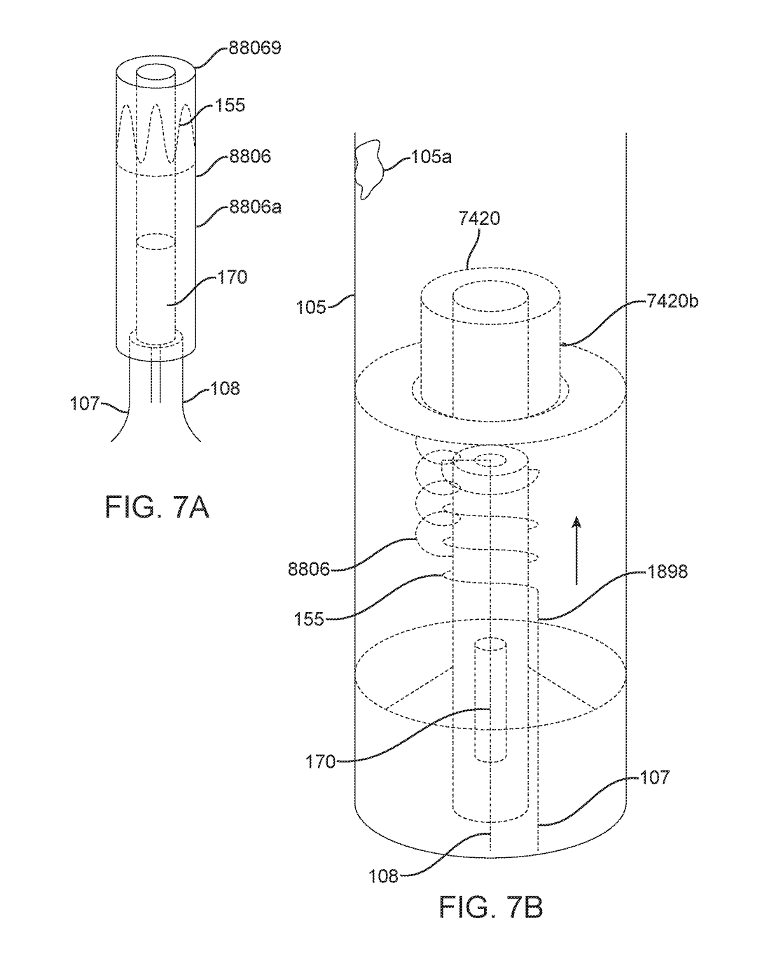

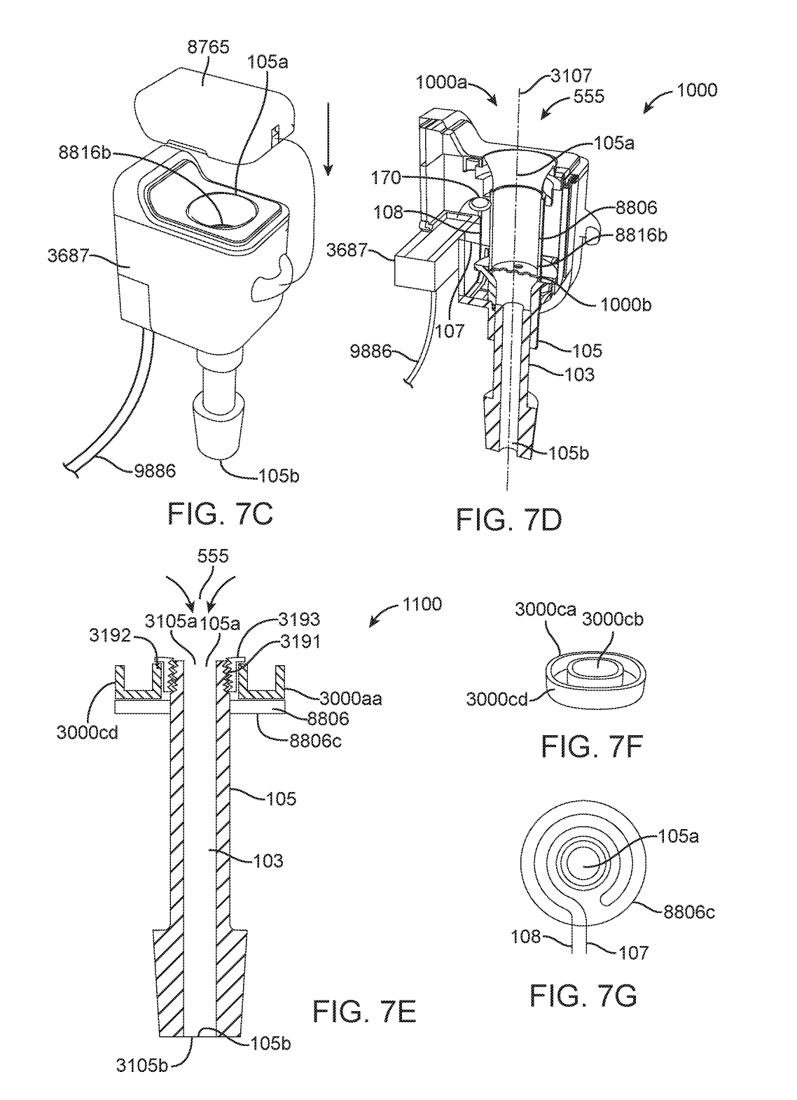

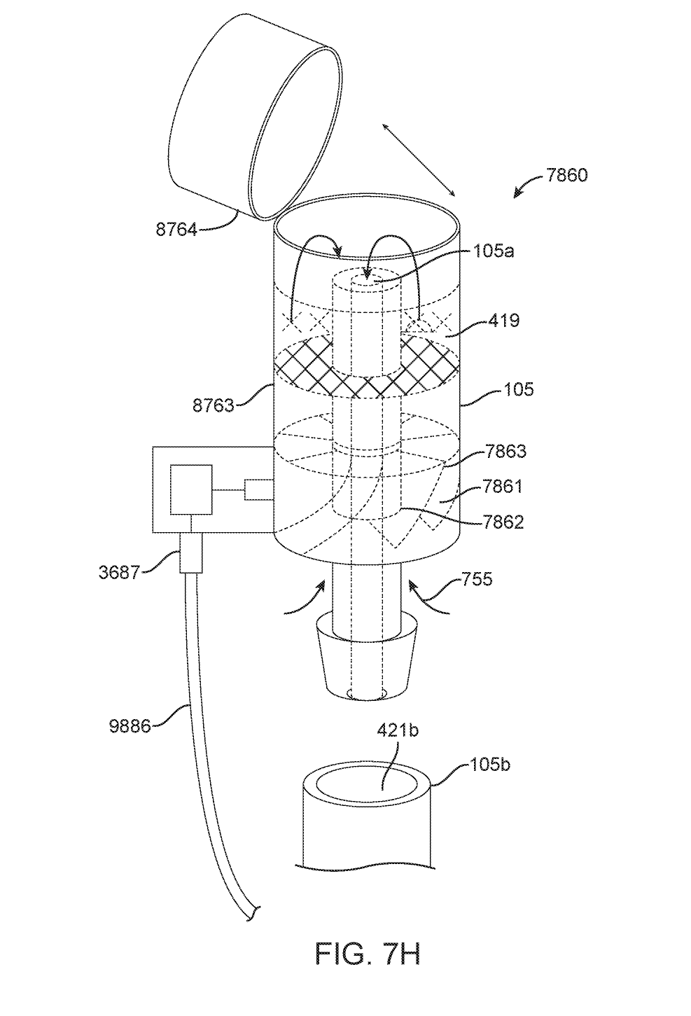

The elongated hollow member 105 as is shown in this embodiment of EVE 2000 is a modified version of a quartz banger as is known in the art. This elongated hollow member has a substantially enclosed dish, proximate the first end 105a, with a protrusion in the center, with the phyto material contact surface 7420 facing the first end 105a and the second side phyto material contact surface 7420b as an inside of this protrusion where within is disposed the heating element 8806 in the form of a ceramic rod heating element 8806a (FIG. 7A) and the temperature sensor 170. The heating element 8806 heats the modified version of a quartz banger from a bottom thereof and the phyto material extract 419 is applied to an opposite side of the protrusion from the heating element 8806 and temperature sensor 170. The phyto material extract 419 is heated by thermal energy that propagates through the glass and is used to vaporize the phyto material 419 at the predetermined temperature for the vapors thereof for being inhaled through the fluid pathway 103. As is shown in an uncoupled orientation, the heating element housing 8806a is not frictionally engaged with the elongated hollow member.

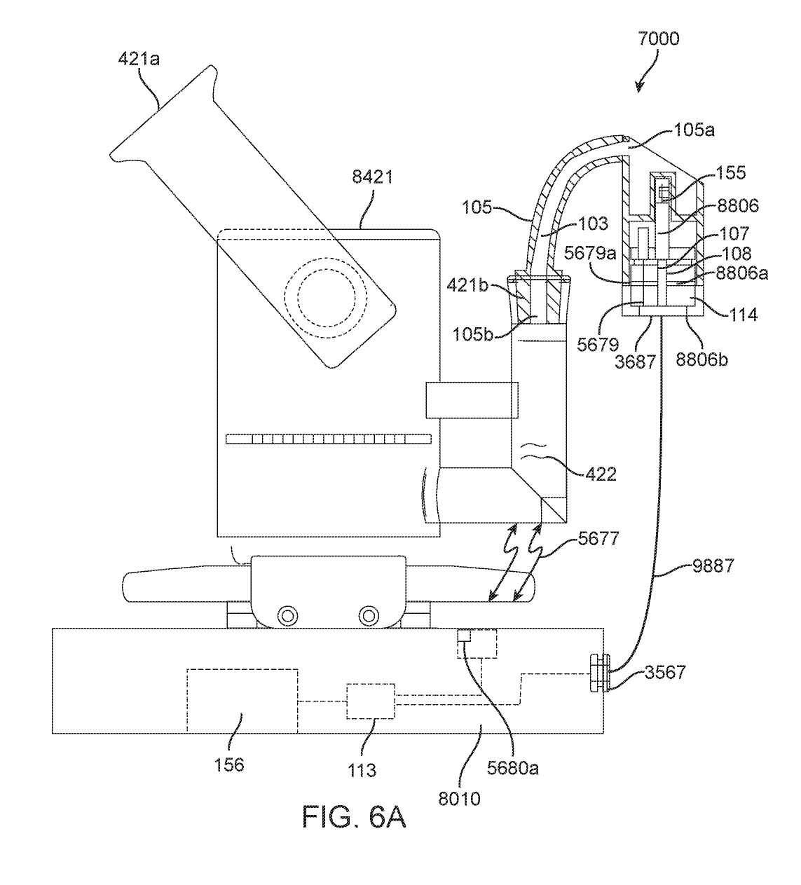

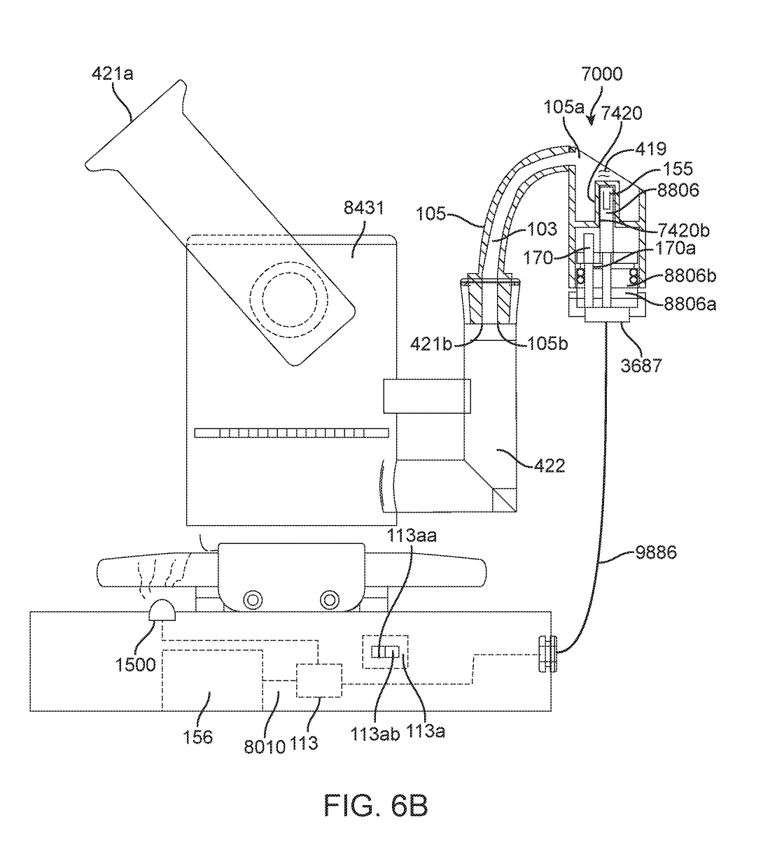

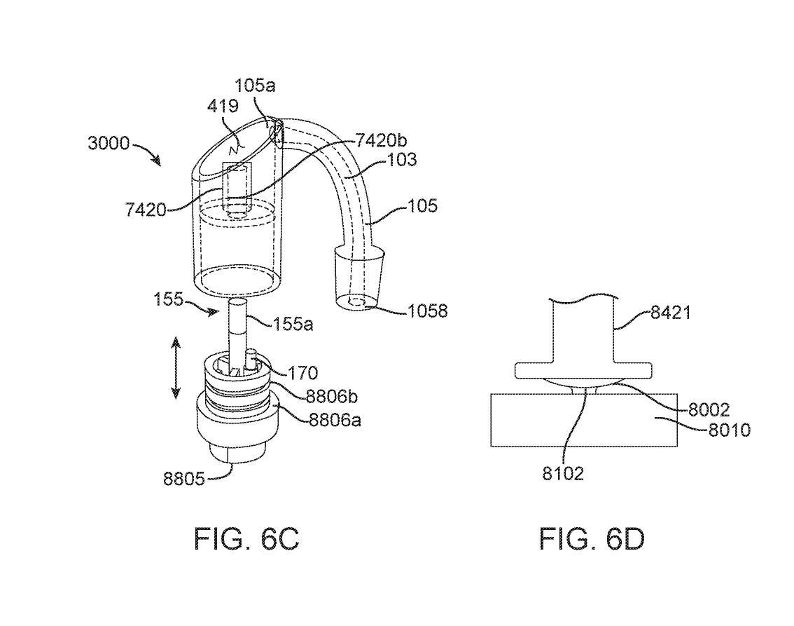

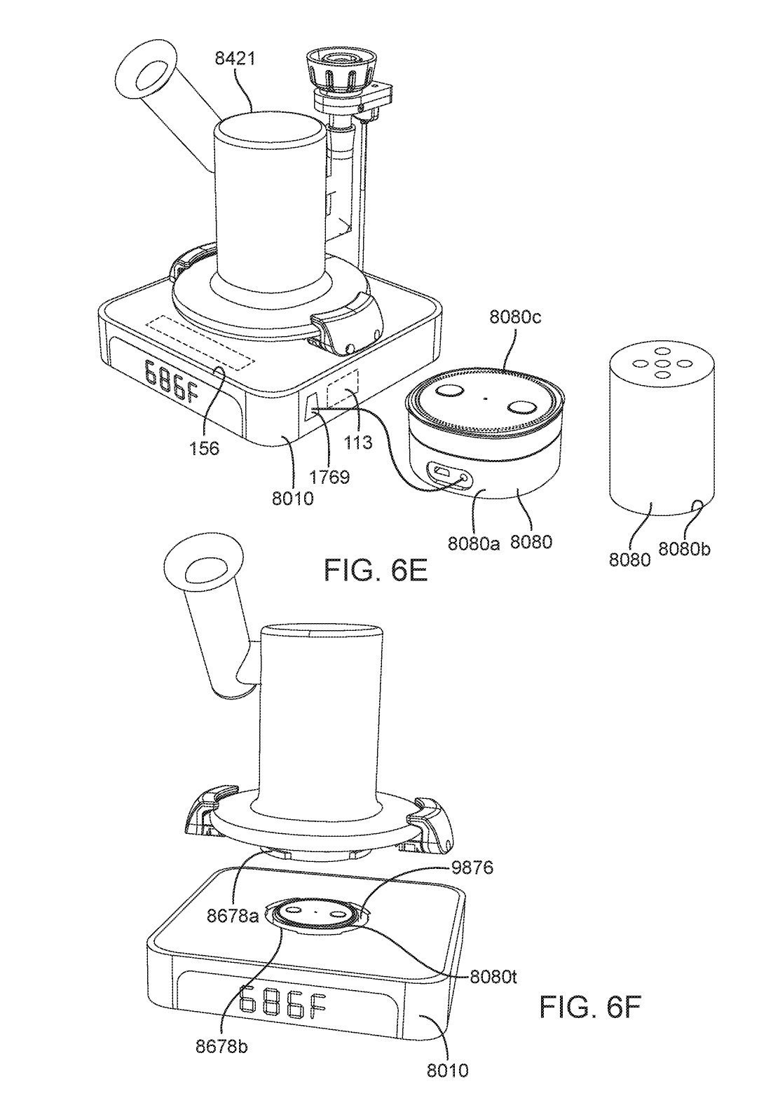

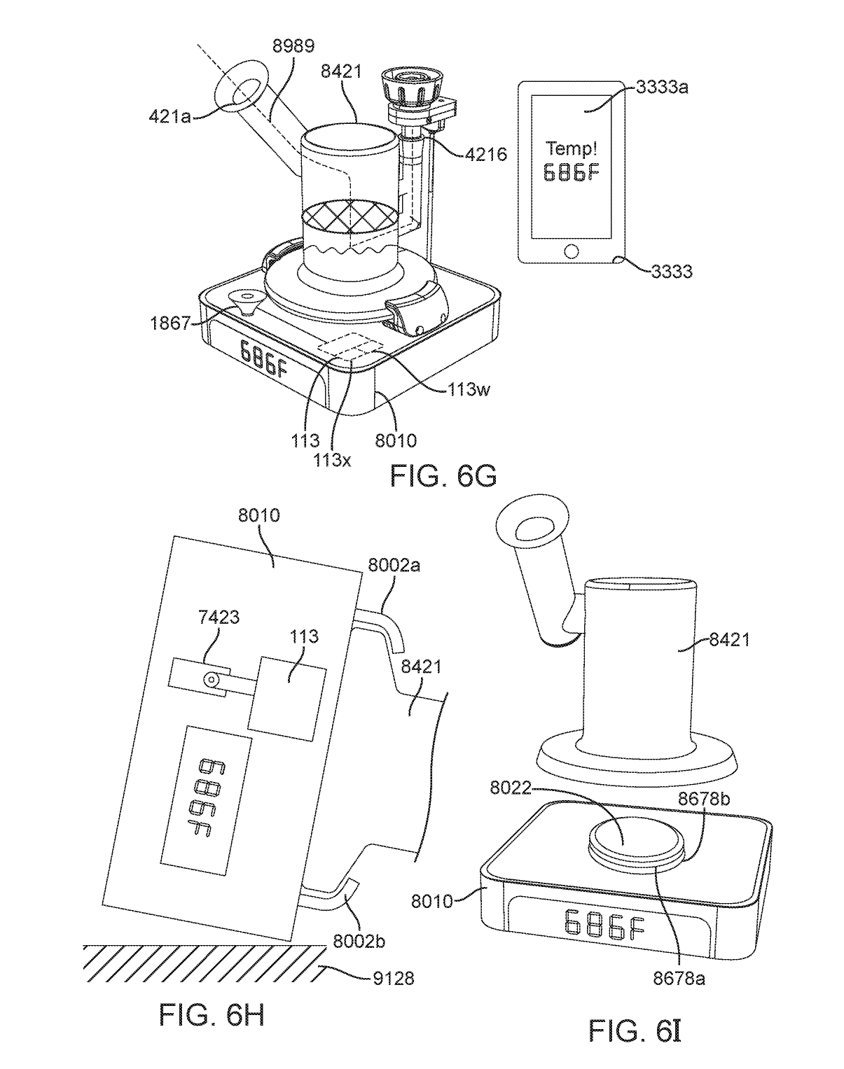

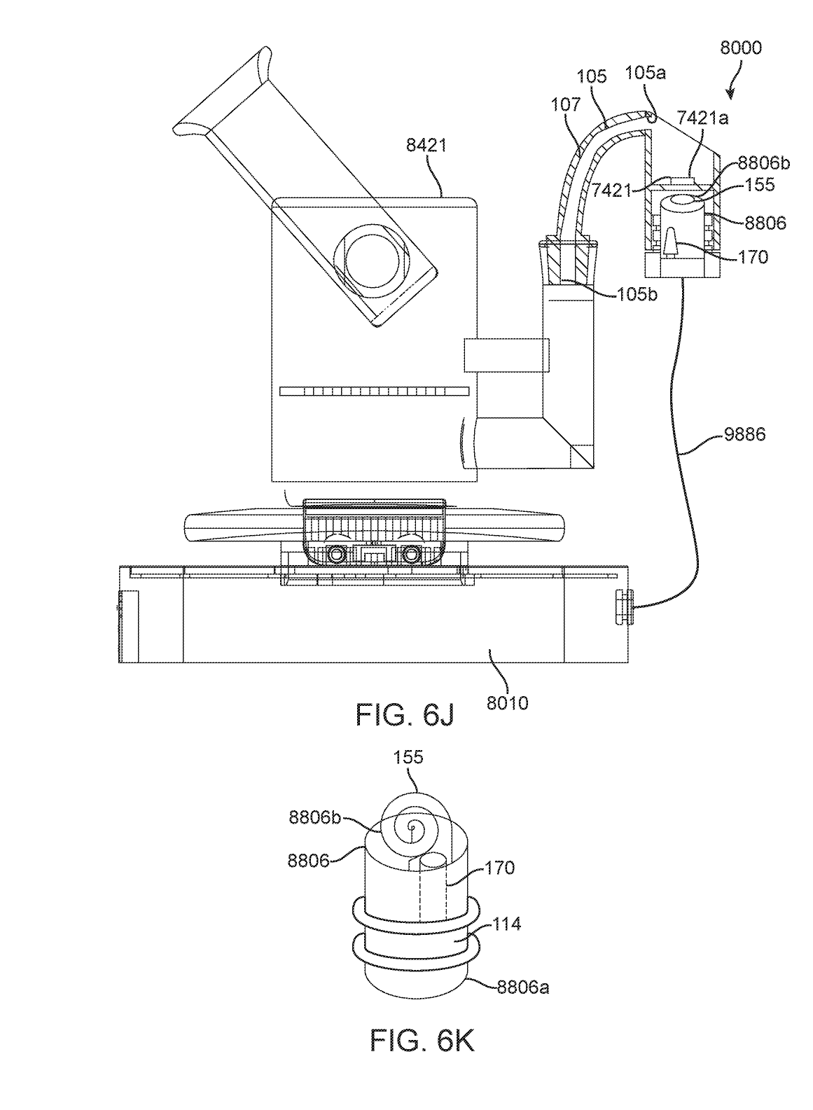

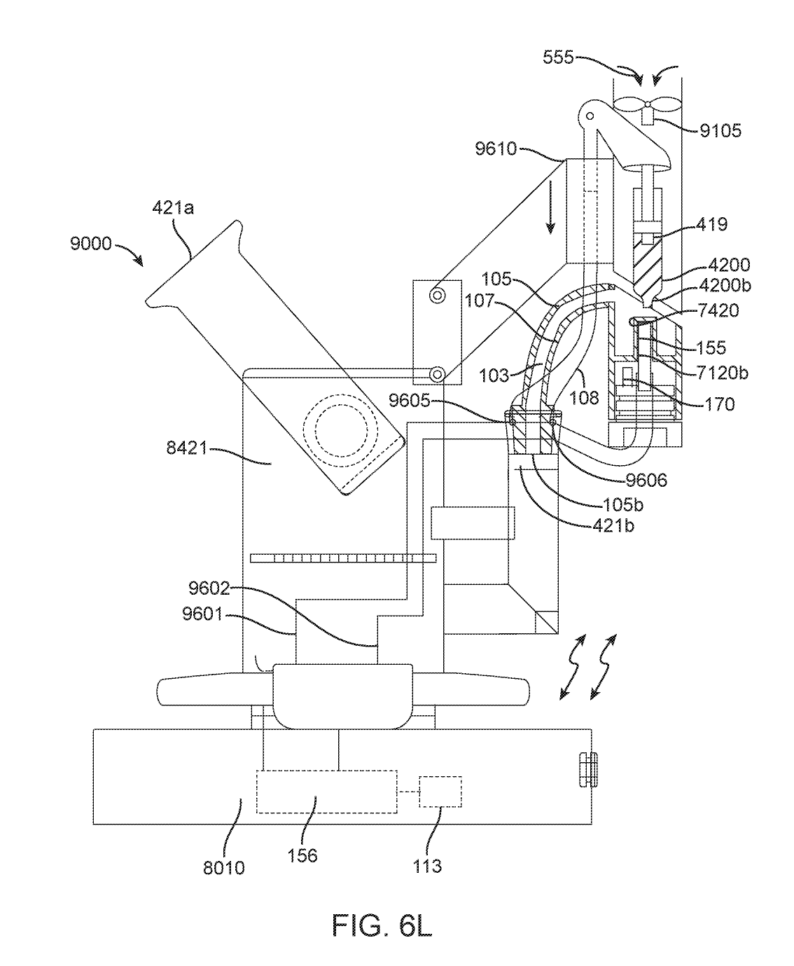

Typically the predetermined temperature is between 300 degrees Fahrenheit and 700 degrees Fahrenheit, where phyto material extracts 419 have a higher predetermined temperature than the phyto material 420, in the form of leaf, where these have a predetermined temperature that is lower than 440 degrees Fahrenheit. FIG. 6C also explains details about the EVE 2000.

Referring back to FIGS. 4D and 4E, the frictional engagement mechanism 8002 is shown in more details, wherein FIG. 4D illustrates the frictional engagement mechanism 8002 from a side view and FIG. 4E from a top view. The frictional engagement mechanism 8002 is formed from an adjustable clamping mechanism 1002 that includes a first jaw 1002a and a second jaw 1002b disposed opposite the first jaw 1002a for respectively sliding within a first track 1401 and a second track 1402.

As is shown in FIG. 4D, a releasable locking mechanism 8123 is coupled with the first jaw 1002a and the second jaw 1002b, wherein a separation between the first jaw and the second jaw is decreased for frictionally engaging of the water pipe 421 (as shown in FIG. 4B) and wherein the releasable locking mechanism 8123 is locked in place to secure the frictional engagement of the water pipe 8421 by the first and second jaws, 1002a and 1002b, and wherein the releasable locking mechanism 8123 is unlocked and a separation between the first jaw and the second jaw is increased for frictionally disengaging of the water pipe 421. The releasable locking mechanism 8123 is envisaged as being a such as those use in zip ties, where in a single direction the mechanism locks, when the jaws 1002a and 1002b have their separation decreased and prior to the jaws 1002a and 1002b having their separation increased, the locking mechanism 8123 is released, such as is the case with a releasable zip tie.

In order to release the locking mechanism 8123 in accordance with this embodiment of the invention, the locking mechanism 8123 is pulled upwards and ratchet teeth 8123a disengage from locking rails as shown in FIG. 4D, this enabling of the jaws 1002a and 1002b to be slid apart. The first jaw 1002a is coupled with a first free end 1002aa having teeth 8123a for engaging the ratchet teeth 8123a and the second jaw 1002b is coupled with a second free end 1002ba having teeth 8123a for engaging the ratchet teeth 8123a.

FIG. 4E shows the first and second jaws 1002a and 1002b being forced towards each other with the first and second free ends 1002aa and 1002ba both engage the ratchet teeth 8123a and are compressed about the water pipe 8421, preferably about a base thereof, or a side, anywhere where the water pipe 8421 is able to be frictionally engaged with the frictional engagement mechanism 8002. The locking mechanism 8123 prevents the first and second free ends from allowing of the first and second jaws to move apart, until the releasable locking mechanism 8123 is unlocked. Preferably the frictional engagement mechanism 8002 is placed on a base of the water pipe 8421 to provide it with additional stability to the water pipe 8421.

Referring to FIG. 4F, the frictional engagement mechanism 8002 is shown having an adjustable clamping mechanism 8008 having a first jaw 8002a and a second jaw 8002b disposed opposite the first jaw 8002a, the first and second jaws are mechanically coupled to a lead screw 8003, for upon rotating of the lead screw 8003 in a clockwise direction for increasing a frictional engagement of the water pipe 8421 and for upon rotating of the lead screw 8003 in a counter clockwise direction for decreasing the frictional engagement of the water pipe 8421, wherein a spacing between the first jaw 8002a the first and second jaws 8002a and 8002b is decreased. The first and second jaws for respectively sliding within a first track 8401 and a second track 8402.

FIG. 4F shows releasable locking mechanism 8123 in a locked state and FIG. 4G shows releasable locking mechanism in an unlocked state. FIGS. 4A and 4B show the releasable locking mechanism clamped onto the water pipe 8421. In this embodiment, the lead screw 8003 has a fine enough pitch of its thread that it prevents the first and second jaws from accidentally disengaging from the water pipe 8421 once its frictionally engaged.

Further referring to FIG. 4F, optionally a motor 8125 is mechanically coupled to the lead screw 8003 and electrically coupled with the first control circuit 113 for controllably rotating of the lead screw 8003 for rotating in a clockwise direction for increasing the frictional engagement of the water pipe 8421 and for upon rotating of the lead screw 8003 in the counter clockwise direction for decreasing the frictional engagement of the water pipe 8421. Having such a motor 8125 facilitates the frictional engaging of the water pipe 8421 without having to manually turn the lead screw 8003. Potentially using a clutch 8125a is envisaged between the motor 8125 to allow the lead screw 8003 to be moved manually without engaging of the motor 8125.

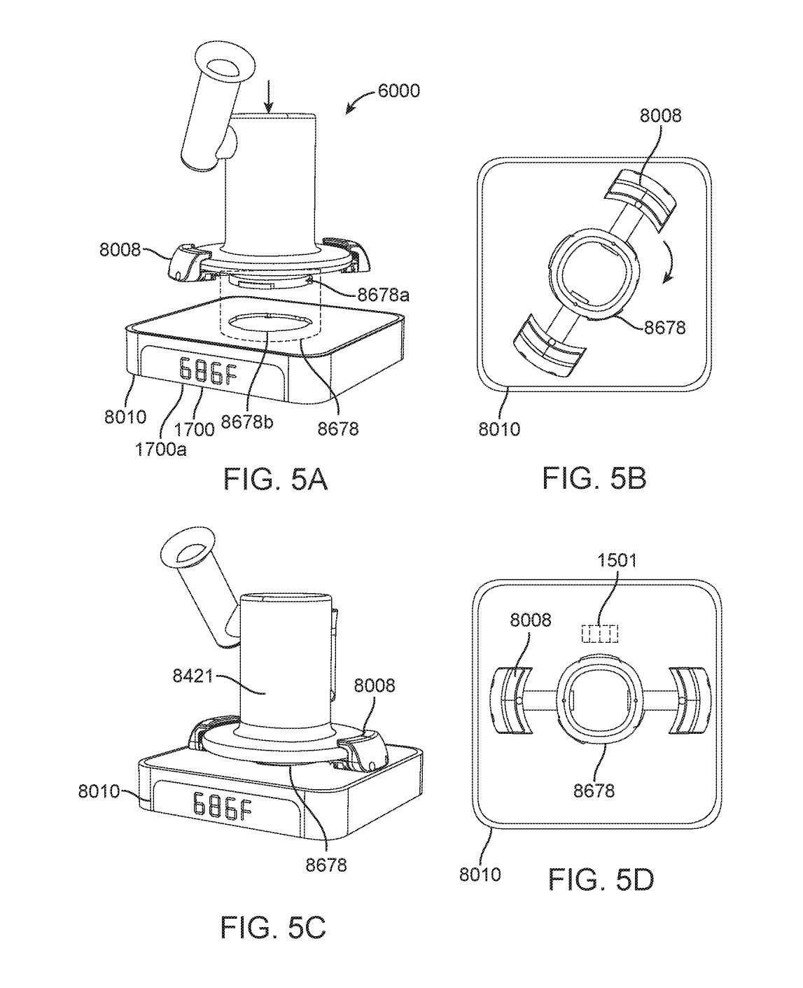

Referring to FIGS. 5A through to 5D, a twist lock coupling 8678 is shown for engaging of the water pipe 8421 with the first housing 8010 in accordance with a sixth embodiment of the invention 6000. The twist lock coupling 8678 is formed from a rotating portion 8678a and a static portion 8678b, the twist lock coupling rotating portion 8678 coupled with the adjustable clamping mechanism 8008 and the static portion 8678b coupled with a first housing 8010, the twist lock coupling 8678 is for operating in locked mode of operation and an unlocked mode of operation.

FIGS. 5A and 5B show the twist lock coupling 8678 in an unlocked mode of operation, where the rotating portion 8678a and a static portion 8678b are other than frictionally engaged and the water pipe is un coupled with the first housing 8010. FIG. 5A shows the twist lock coupling 8678 from a perspective view and FIG. 5B shows the twist lock coupling 8678 from a top view with the water pipe 8421 removed for clarity.

FIGS. 5C and 5D shown the twist lock coupling 8678 in the locked mode of operation, wherein the rotating portion 8678a and the static portion 8678b are frictionally engaged together and the water pipe is coupled with the first housing 8010. FIG. 5C illustrates the locked mode of operation from a perspective view and FIG. 5D illustrates the locked mode of operation from a top view with the water pipe 8412 removed for clarity.

In order to transition from the unlocked mode of operation to the locked mode of operation, preferably the adjustable clamping mechanism 8008 is first frictionally engaged with the water pipe 8421 and then the rotating portion 8678a of the twist lock coupling 8678 is pushed against the first housing 8010 and oriented such that twist lock coupling 8678 is aligned along its predetermined starting orientation, as shown in FIG. 5A and then twisted into place as is shown in FIG. 5B, clockwise in this embodiment.

In the locked mode of operation the water pipe 8421 is releasably coupled with the first housing 8001. As shown in the first and second embodiments of the invention as well as in the FIGS. 5A and 5C, the adjustable clamping mechanism 8008 is for frictionally engaging of the water pipe 421 from a bottom or base thereof, however the scope of the invention is not limited to just the base but to also any part of the water pipe 421 such as a side thereof, as is shown in FIGS. 4A and 4B. As is shown in FIG. 5A, a control panel 1700 comprises a display screen 1700a that is electrically coupled with first control circuit 113. However, frictionally engaging of the side of the water pipe 8421 may result in the water pipe 8421 to be less stable.

Optionally, the frictional engagement mechanism 8003, as is shown in FIGS. 4F and 4G has the adjustable clamping mechanism 8008 formed from the first jaw 8002a and the second jaw 8002b disposed opposite the first jaw 8002a, the first and second jaws mechanically coupled to the lead screw 8003, for upon rotating of the lead screw 8003 in a clockwise direction for increasing a frictional engagement of the water pipe 8421 and for upon rotating of the lead screw in a counter clockwise direction for decreasing a frictional engagement of the water pipe 8421, wherein a spacing between the first jaw 8002a the first and second jaws 8002a and 8002b for respectively sliding within a first track 8401 and a second track 8402. In addition the twist lock coupling 8678 having rotating portion 8678a and a static portion 8678b, the twist lock coupling rotating portion 8678 coupled with the adjustable clamping mechanism 8008 and the static portion 8678b coupled with the first housing 8010, the twist lock coupling 8678 for operating in locked mode of operation and an unlocked mode of operation, in the locked mode of operation the rotating portion 8678a and the static portion 8678b are frictionally engaged together and the water pipe is coupled with the first housing 8010 as is shown in FIG. 4F and in the unlocked mode of operation the rotating portion 8678a and a static portion 8678b are other than frictionally engaged and the water pipe is uncoupled with the first housing 8010, as shown in FIG. 4G.

Using the twist lock coupling 8678 allows for the water pipe 8421 to be removed from its base, first housing 8010, so that it can be filled with water and easily cleaned. This also allows for the base industrial design to include the static portion 8678b and not the adjustable clamping mechanism 8008. So this allows for various adjustable clamping mechanism 8008 to be envisaged for frictionally engaging a plurality of different shaped water pipes 8421, such as beaker or Erlenmeyer and various water pipes geometries so that a single base, first housing 8010, with the twist lock coupling can be used with various water pipe shapes. Other frictional engagement mechanisms are also envisaged that do not use a lead screw for adjusting the frictional engagement and perhaps a set screw as the releasable locking mechanism 8123.

Referring to FIG. 6D, the frictional engagement mechanism 8002 is in the form of a suction cup 8102 or sticker or a zip tie or other fastening system for frictionally engaging of the water pipe 8421 with the frictional engagement mechanism 8002 in an engaged state. The suction cup is potentially envisaged as being an active device whereby the water pipe 8421 is placed in proximity thereto and then a button is pressed and the water pipe 8421 is sucked onto the suction cup 8102.

Referring to FIG. 6I an adhesive tape 8022 is also envisaged for adhering of the water pipe 8421 to the rotating portion 8678a and as such a clamping mechanism (as shown in FIGS. 4D and 4F) is not necessary for adhering of the water pipe 8421 to the first housing 8010. Optionally the water pipe 8421 is adhered directly to the first housing 8010, however this does not allow it to be easily removed therefrom.

Referring to FIG. 6A, a second control circuit 114 is disposed as part of the EVE 7000, in accordance with a seventh embodiment of the invention and electrically coupled with the heating element 8806 first and second electrical contacts 107 and 108, of the resistive heater 155 and having a power coupling input port 3687 and a second wireless transceiver 5679 as part of the second control 114 circuit. The first control circuit 113 is disposed within the first housing 8010 and the second control circuit 114 is electrically coupled with the electrical power source 156 and comprising a power coupling output port 3567 and a first wireless transceiver 5680.

An electronic vaporization element coupling cable 9887 is provided for electrically coupling of the first control circuit 113 to the second control circuit 114 and the first wireless transceiver 5680 for communicating with the second wireless transceiver 5679 through a wireless communication link 5677, whereby the electronic vaporization element coupling cable 9886 provides electrical power to the second control circuit 114 and the wireless communication link 5677 is for exchanging a control data between the first and second control circuits 113 and 114 for at least one of heating and maintaining of the heating element 8806 at the predetermined temperature and disabling heating of the heating element 8806.

Optionally, the first wireless transceiver 5680 comprises a first optical transceiver 5680a and the second wireless transceiver 5679 comprises a second optical transceiver 5679a as the wireless communication link 5677 wherein the control data between the first and second control circuits 113 and 114 is transmitted optically for at least one of heating and maintaining of the phyto material 419 at the predetermined temperature and disabling heating of the heating element 8806. Other wireless technologies are also envisaged, such as radio frequency. In this embodiment with the wireless communication link 5677, the electronic vaporization element coupling cable 9886 is a dual conductor for carrying of electrical power from the electrical power source 156 to the EVE 3000.

Typically, the water pipe 8421 is made from glass and as such it facilitates a transmission of optical signals therethrough. So in the case of the first optical transceiver 5680a and the second optical transceiver 5679a, preferably infrared LEDs and infrared receivers are used for each of the transceivers. Infrared communication is preferred over wireless, such as Bluetooth®, because it is cheaper and pairing is not necessary between the two control circuits as well as infrared propagates very well in a home environment and is low power.

Referring to FIG. 6B and to EVE 3000, a first temperature sensor 170 is provided in thermal communication with the heating element 8806, the first temperature sensor 170 comprising a temperature signal output port 170a and for generating a temperature signal in dependence upon a temperature of the heating element 8806. The electronic vaporization element coupling cable 9886 for electrically coupling of the first control circuit 113 to the heating element 8806 first electrical contact 107 and a second electrical contact 108 and the temperature signal output port 170a, wherein the first control circuit 113 is for receiving of the temperature signal and for pulse width modulating electrical power provided to the resistive heater 155 along the electronic vaporization element coupling cable 9886 from the electrical power source 156 for at least one of heating and maintaining of the phyto material at the predetermined temperature and disabling electrical power provided to the heating element 8806.

In this case the electronic vaporization element coupling cable 9886 is at least a three-conductor cable, carrying ground a positive voltage and the temperature signal from the first temperature sensor 170 to the first control circuit 113 and electrical power from the electrical power source 156 to the EVE 3000. The electronic vaporization element coupling cable 9887 as shown in FIG. 6A is only two-conductor cable that carries ground and positive voltage to the second control circuit 114.

Referring to FIG. 6B and to FIG. 6C, a phyto material contact surface 7420 is shown disposed between the resistive heater 155 and the phyto material extract 419, the phyto material contact surface 7420 for receiving of thermal energy from the resistive heater 155 on a second side thereof 7420b and for transmitting at least a portion of the receive thermal energy into the phyto material 419 disposed on the phyto material contact surface 7420 for the at least one of heating and maintaining of the phyto material 419 at the predetermined temperature.

Referring to FIG. 6C, in this embodiment of EVE 3000, the phyto material contact surface 7420 comprises glass and the resistive heater 155 comprises a ceramic heater 155a, where the ceramic heater 155a heats the phyto material extract 419 through the glass phyto material contact surface 7420 where the phyto material does not contact the ceramic heater 155a directly.

Furthermore, the heating element 8806 is releasably coupled with the elongated hollow member 105 proximate the first end 105a using a frictional coupling 8805. The heating element 8806 is coupled with a heating element housing 8806a and for the frictional coupling at least a silicone rubber O-ring 8806b is disposed about the heating element housing 8806a and the silicone rubber O-ring 8806b frictionally engaged at portion of the elongated hollow member 105. This allows for the heating element housing 8806a to be inserted proximate the phyto material contact surface 7420 for having the heating element 8806 provide of thermal energy to the glass phyto material contact surface 7420 phyto material contact surface 7420.

Optionally the phyto material contact surface 7420 is formed from ceramic and the elongated hollow member 105 comprises ceramic. Selecting a low thermal conductivity material is preferable for the construction of the elongated hollow member 105 as this reduces thermal energy transfer from the glass phyto material contact surface 7420 to other parts of the elongated member 105. Also having the elongated hollow member 105 to be releasably coupled with the heating element housing 8806a allows for easy cleaning of the elongated member 105 and the phyto material contact surface 7420 as it can be cleaned using isopropyl alcohol and therefore does not require cleaning of the heating element 8806 and the first temperature sensor 170.

Referring to FIG. 6B the first temperature sensor 170 disposed proximate the heating element 8806 and the second side of phyto material contact surface 7420b and in thermal communication therewith, the first temperature sensor 170 having the temperature signal output port 170a for generating the temperature signal, the first control circuit 113 for generating a first temperature signal data 113ab from the temperature signal in dependence upon a temperature of the heating element 8806. The first control circuit 113 comprises a first lookup table 113a, wherein the first lookup table 113a comprises at least a calibration value 113aa for correlating the predetermined temperature with the first temperature signal data 113ab.

Having the first lookup table 113a facilitates calibration of temperature signal with an actual temperature of the heating element 8806. The first temperature sensor 170 is measuring a temperature in proximity of the heating element 8806, however the predetermined temperature is important as this is the temperature at which the phyto material extract is being heated and is the temperature of the phyto material contact surface 7420. Therefore its preferable to determine the predetermined temperature at the phyto material contact surface 7420 for the phyto material 419 disposed thereon.

For example, the first temperature sensor 170 will read a temperature that is lower then an actual temperature of the heating element 8806 and the first side of the phyto material contact surface 7420b may be at a higher temperature. With a process of measuring an actual temperature of the phyto material contact surface 7420b and the temperature signal data 113ab, at least a calibration value 113aa is generated for correlating the temperatures to extrapolate through the first lookup table 113a the temperature of the phyto material contact surface 7420b when in use. Preferably this calibration is performed in advance.

In addition a LED 1500 electrically coupled with first control circuit 113 and protruding past the first housing 8010 for illuminating of the water pipe. Referring to FIG. 5D, optionally a LED display 1501 comprising a plurality of three color light emitters arranged in a two dimensional matrix for being electrically coupled with first control circuit 113 for illuminating the water pipe, such as a colored OLED display. This then facilitates changing colors and other images to be used for illuminating of the water pipe 8421. A laser emitter is also potentially envisaged for illuminating the water pipe 8421 and for a portion of this light to be reflected and refracted by the water pipe 8421 for creating an interesting visual display for entertainment purposes.

Referring to FIG. 6E, a voice recognition processor 8080, for example an Alexa Voice Services (AVS) 8080a or a Google® Home Voice Services 8080b, as is known to a person of skill in the art is shown one of electrically and wirelessly coupled with the first control circuit 113, the voice recognition processor 8080 for receiving of voice commands from a user for at least one of controlling the heating of the phyto material extracts 419 to the predetermined temperature and for adjusting of the predetermined temperature. Optionally, the voice recognition processor 8080 is electrically powered by the electrical power source 156 through a power output port 1769, such as a USB port.

For example the user comes home and says “Alexa, Big E 650 degrees Fahrenheit” and AVS 8080a processes the command and instructs the first control circuit 113 to enable heating of the heating element 8806 to the predetermined temperature where the phyto material contact surface is at approximate at 650 degrees Fahrenheit. With such commands this would then allow for almost full control of this embodiment of the invention, thereby allowing a user that is for example with limited mobility to be able to use this embodiment of the invention without the need for much manual input.

Referring to FIG. 6F, optionally a cavity 9876 is formed within the first housing 8010 for receiving of the voice recognition processor 8080 therein. The voice recognition processor 8080 comprises at least one LED 8080z and the at least one LED 8080z is for illuminating of at least a portion of the water pipe through optical reflection and refraction. The first housing 8010 and water pipe 8421 are arranged in such an orientation that microphones disposed as part of the functionality of the voice recognition processor 8080 are not covered up significantly for allowing of the voice recognition processor 8080 to receive of verbal commands.

Referring to FIG. 6G, optionally the first control circuit 113 comprises at lease one of a WIFI module 113w electrically coupled therewith for communicating with the internet for at least one controlling the heating of the phyto material to the predetermined temperature and for adjusting of the predetermined temperature through the internet and a Bluetooth module 113x for communicating with a smartphone 3333 having a smartphone application 3333a for being executed therein, wherein the smartphone application communicates with the first control circuit 113 through the Bluetooth® module 113x for at least one controlling the heating of the phyto material to the predetermined temperature and for adjusting of the predetermined temperature.

Further optionally, a speaker 1867 is disposed within the first housing 8001, the speaker 1867 electrically coupled with the first control circuit 113 and is for optionally being coupled to the Internet or to the Smartphone 3333 for having music streamed thereto. As is also shown in the FIG. 6C, the water pipe 8421 has the input port 421b and an inhalation aperture 421a with the water pipe fluid pathway 8989 formed therebetween

Referring to FIG. 6H, a tilt sensor 7423 is electrically coupled with the first control circuit 113 for determining whether the first housing 8010 has become inverted in relation to ground 9123 and for disabling the heating element 8806 for heating the phyto material to the predetermined temperature. Only a lower portion of the water pipe 8421 is shown in this figure and the rest has been cut off for clarity.

Referring to FIG. 6j and to EVE 8000 in accordance with an eight embodiment of the invention, a first temperature sensor 170 is provided in thermal communication with the heating element 8806, the first temperature sensor 170 comprising a temperature signal output port 170a and for generating a temperature signal in dependence upon a temperature of the heating element 8806. The electronic vaporization element coupling cable 9886 is for electrically coupling of the first control circuit 113 to the heating element 8806 first electrical contact 107 and a second electrical contact 108 and the temperature signal output port 170a, wherein the first control circuit 113 is for receiving of the temperature signal and for pulse width modulating electrical power provided to the resistive heater 155 along the electronic vaporization element coupling cable 9886 from the electrical power source 156 for at least one of heating and maintaining of the phyto material at the predetermined temperature. In this case the electronic vaporization element coupling cable 9886 is at least a three-conductor cable, carrying ground a positive voltage and the temperature signal from the first temperature sensor 170 to the first control circuit 113 and electrical power from the electrical power source 156 to the EVE 8000.

In this embodiment of EVE 8000, the resistive heater 155 is in the form of a spiral or a pancake coil heater 8806b, as is detailed in FIG. 6K. A phyto material contact surface 7421 is shown disposed between the resistive heater 155 in the form of the coil heater 8806b and the phyto material extract 419, the phyto material contact surface 7420 for receiving of thermal energy from the resistive heater 155 on a second side thereof 7421b and for transmitting at least a portion of the receive thermal energy into the phyto material 419 disposed at a first side thereof 7421a for the at least one of heating and maintaining of the phyto material 419 at the predetermined temperature. Such a heater provides for a large contact surface for the phyto material contact surface 7420.

FIG. 6L illustrates a ninth embodiment of the invention 9000, where a first electrical power rain 9601 and a second electrical power rail 9602 are releasably electrically coupled with a first power coupling 9603 and a second power coupling 9604 to the first control circuit 113 and to the electrical power source 156. The first and second power couplings allow for electrical power from the electrical power source 156 to be coupled to the water pipe 8421. The first electrical power rails 9601 and a second electrical power rail 9602 are disposed about the water pipe 8421 or optionally embedded into the glass of the water pipe 8421. The first and second electrical power rails 9601 and 9602 terminate proximate the water pipe input port 421b at first rail power port 9605 and a second rail power port 9606, where these ports then coupled with the first electrical contact 107 and the second electrical contact 108 of the heating element 8806. Preferably this coupling is a magnetic and releasable coupling. Embedding the a first electrical power rain 9601 and a second electrical power rail 9602 within the glass advantageously allows for cleaner looking interface between the EVE in accordance with the embodiments of the invention as there are less wires hanging from the EVE. Potentially it is also envisaged to have the water pipe 8421 portions that are manufactured from vanadium dioxide, which can possible allow for the conduction of electricity but not the conduction of heat.

In addition, a syringe actuator 9610 is electrically coupled with the first rail power port 9605 and the second rail power port 9606, The syringe actuator is for actuating a syringe 4200 that is filled with phyto material extract 419 for depositing the phyto material extract 419 in a predetermined volume onto the phyto material contact surface 7420 from a phyto material extract output port 4200a. In this embodiment, ambient air 555 enters into the first end 105a of the elongated member 105 through an ambient air input aperture 555a disposed upstream and in fluid communication with a mass airflow meter 9105, disposed downstream thereof and which measures the mass of air substance which passes therethrough per unit of time, electrically coupled with the first control circuit 113 through the first rail power port 9605 and the second rail power port 9606 and for receiving of ambient air 555 passing therethrough the and for measuring a predetermined mass of air passing therethrough as a first air mass, the mass airflow meter 9105 for generating a first air mass signal in dependence upon an initial flow of ambient air passing therethrough and for generating a first air mass data based on the mass of air passing therethrough. The first mass air flow data being wirelessly communicated with the first control circuit 113 using the a third wireless transceiver 5678 for communicating with the first wireless transceiver 5680 and the syringe actuator 9610 comprising a fourth wireless transceiver 5677 for communicating with the first wireless transceiver 5680.

In use, a significant majority if ambient air 555 that enters into the first end 105a of the elongated member 105 enters through the ambient air input aperture 555a, where its mass is measured and simultaneously the phyto material 419 is extruded from the syringe 4200 and this phyto material extract 419 is vaporized and inhaled from the inhalation aperture 421a. Based on the predetermined volume of phyto material extract 419 that is extruded and based on a first air mass data, a measured dose system is envisaged, where the mass of ambient air entering the system is known as well as the predetermined volume that is disposed onto the phyto material contact surface 7420. Of course calibrating of the system is necessary to determine a percentage of phyto material vapor being present in the mass of air flowing through the mass airflow meter 9105 in time when inhaled from the inhalation aperture 421a. The first control circuit 113 for processing of the first air mass data and for at least one of controlling of the predetermined volume of the phyto material extract 419 being deposited onto the phyto material contact surface 7420 and for controlling of the predetermined temperature through pulse width modulation of electrical power being applied to the heating element 8806.

Referring to FIG. 7A, a heating element 8806 is shown as a tubular heating element with the first temperature sensor 170 disposed inside of the heating element 8806 and the resistive heater 155 is disposed proximate a first end 8806q thereof. Optionally, as is shown in FIG. 7B, the resistive heater 155 is formed from the resistive heater 155 wrapped about a ceramic tube 1898 and forming a tubular heating element. The elongated member 105 and the phyto material contact surface 7420 and having disposed opposite thereof a second side phyto material contact surface 7420b are shown.

FIGS. 7C and 7D illustrate the EVE in accordance with a tenth embodiment of the invention as 1000 where the heating element 8806 is in the form of a ceramic cup heating element in thermal communication with the temperature sensor 170. The EVE 1000 is also termed a leaf attachment, where it is formed from a substantially enclosed housing having the ceramic cup heating element 8816b having an open end 1000a for receiving of the phyto material 420, optionally for receiving also of phyto material extract 419, and a perforated end 1000b having a screen for containing the phyto material 420 therein but allowing ambient air 555 and vaporized phyto material to propagate therethrough. A fluid pathway 3103 propagates from the ceramic cup heating element 8816b having the open end 8816ba to the perorated end 8816bc where the phyto material is inserted therebetween. The perforations prevent at least a major amount of the phyto material 420 from falling through the holes therein.

The heating element 8816b heats the phyto material 420 from the sides. The first end 3105a is proximate open end 8816ba and has a lid 8765 thereon for loading of the phyto material 419 into the heating element 8816b. Thermal energy propagates from the heating element 8816b into the phyto material 420 and generates vapors therefrom for the vapors thereof and ambient air mix for being inhaled through the perorated end 8816bc along the fluid pathway 3103.

Referring to FIG. 7E, the EVE 1100 is in the form of an eleventh embodiment of the invention, a removable cup EVE 1100, wherein the removable cup EVE 1100 comprise a removable cup 3000ca that is made from either ceramic or glass or gold or platinum or silver and that is in thermal communication with a annular heater 8806c. The removable cup 3000ca is in the form of a cutaway torus shape (FIG. 7F) having a center hole 3000cb and a sidewall 3000cd about its circumference. The fluid pathway 3103 propagates from the first end 3105a to the second end 3105b thereof and through the center hole 3000cb.

A threaded coupling 3191 having a spring 3192 and for thermal expansion along the fluid pathway 3103 and a hollow nut 3193 are provided. When assembling, the removable cup 3000ca is disposed on top of the annular heater 8806c and the spring 3192 are hollow nut 3193 engage as part of the threaded coupling 3191 and press the removable cup 3000ca against the annular heater 8806c. The annular heater 8806c heats the removable cup 3000ca and then from an opposite side to the heater the phyto material extract 419 is applied and vaporized and these vapors and ambient air 555 propagate through the center of the hollow nut and through the center of the removable cup 3000ca and through the center of the annular heater 8806c and out through the second end 3105b in the water pipe input port 421b. FIG. 7G illustrates the annular heater 8806c from a top view.

FIG. 7H illustrate the EVE 1200 in accordance with a twelfth embodiment of the invention as 1200 where the heating element 8806 is in the form of a convection heating element 7861 in thermal communication with the temperature sensor 170 and having a ceramic heater core 7862 coupled with a plurality of fins 7863. The plurality of fins 7863 are for being hated by the ceramic heater core 7862 and for heating incoming ambient air 555, which is heated air, then flows over the phyto material 420 that is separated from the plurality of fins by a screen 8763 and it then flows into the first end 105a of the elongated member 105, proximate the removable lid 8764.

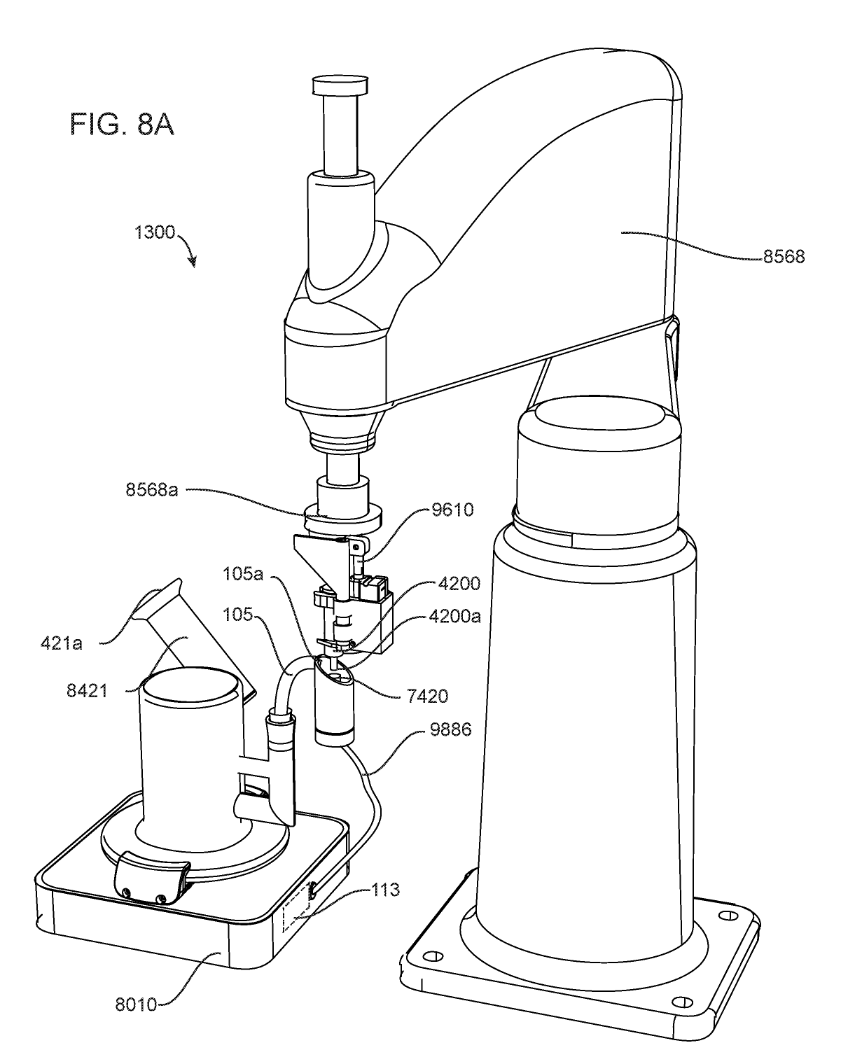

FIG. 8A illustrates a robotic measured dose apparatus 1300 in accordance with a thirteenth embodiment of the invention. The robotic measured dose apparatus 1300 is comprised of a robotic arm 8568 having a plurality of controllable axes, such as at least two axes, as is well known in the art, such as a SCARA robotic arm. The Robotic arm 8568 includes an end effector 8568a. As is shown in FIG. 8A, a syringe 4200 is filled with the phyto material extract 419 and has a phyto material extract output port 4200a. A syringe actuator 9610 is electrically coupled with the first control circuit 113 and mechanically coupled with the syringe 4200 for actuating the syringe 4200 for depositing a predetermined volume of the phyto material extract 419 onto the phyto material contact surface 7420 from the phyto material extract output port 4200a. The robotic arm 8568 comprising the end effector 8568a is coupled with the syringe actuator 9610, where the robotic arm 8568 for controllably positioning of the phyto material extract output port 4200a proximate the phyto material contact surface 7420 for depositing a predetermined volume of the phyto material extract 419 onto the phyto material contact surface 7420 for vaporization of the phyto material extract thereon. Combined with the voice recognition processor 8080 (FIG. 6E) this is a system that allows for complete hands free operation by the end user. Advantageously such a robotic measured dose apparatus 8568 allows for the use of the EVE in accordance with the embodiments of the invention by disabled people who do not have sufficient movement of their limbs to be able to consume the phyto material extracts 419 as needed as part of their medication. The phyto material extracts 419 are pre-loaded into the syringe 4200 and the end effector 8568a positions the phyto material extract output port 4200a to momentarily dispense the phyto material extract 419 for the vaporization and then moves away for potential carb cap operation that is also potentially performed via the robotic arm 8568. A carb cap is not shown in this figure, but the operation thereof is know to those of skill in the art.

Having a device for vaporization of phyto material extracts in accordance with the embodiments of the invention allows for a reduction in potential harm from combustion of the phyto material extracts 419 and phyto materials 420. Furthermore it allows for a portable device that overcomes the deficiencies in the prior art. Having the elongated member of the EVE manufactured from ceramic or glass or quartz allows for easy cleaning. Also because the EVE is manufactured from a low thermal conductivity material it allows for the second end 105b thereof to be substantially cooler than the first end 105a, thus allowing the elongated hollow member 105 to provide additional cooling to the vapors 421 and ambient air mix 555 when propagating therethrough. Ceramic and glass materials are also easy to clean and do not typically stain when used for vaporization of phyto material extracts 419. The LED advantageously provides for an indication to the end user of the approximate temperature of the predetermined temperature of the EVE. Preferably the electrical power source 156 is from internal battery power, however a wall adapter is also envisaged.

Numerous other embodiments are envisaged without departing from the spirit or scope of the invention.

What I claim is:

1. A method and device for vaporizing phyto material for frictionally engaging a water pipe having an input port and an inhalation aperture with a water pipe fluid pathway formed therebetween comprising:
a electronic vaporization element (EVE) comprising:
an elongated hollow member having a first end and a second end opposite the first end, a fluid pathway propagating from the first end to the second end thereof, the second end for coupling with the water pipe input port, and
a heating element disposed proximate the first end and comprising a first electrical contact and a second electrical contact the heating element comprising a resistive heater disposed between the first and second electrical contacts,
the resistive heater for heating the phyto material disposed onto a phyto material contact surface to a predetermined temperature for vaporizing of the phyto material for creating a vapor therefrom and upon inhalation from the inhalation aperture this vapor is mixed with ambient air and flows through the fluid pathway from the first end and propagates through the input port of the water pipe and through to the inhalation aperture;
a removable electrical power source comprising a first housing for having an electrical power source contained therein, the first housing comprising a frictional engagement mechanism for frictionally engaging of the water pipe; and,
a first control circuit disposed within the first housing and electrically coupled with the electrical power source and the first and second electrical contacts of the EVE with an electronic vaporization element coupling cable electrically disposed therebetween, the first control circuit for providing electrical power from the electrical power source to the heating element for heating the phyto material disposed onto the phyto material contact surface to the predetermined temperature.

2. A method and device for vaporizing phyto material according to claim 1 wherein the frictional engagement mechanism 8002 comprises:
an adjustable clamping mechanism comprises a first jaw and a second jaw disposed opposite the first jaw for respectively sliding within a first track and a second track; electronic vaporization element coupling cable; and
a releasable locking mechanism coupled with the first jaw and the second jaw, wherein a separation between the first jaw and the second jaw is decreased for frictionally engaging of the water pipe and wherein the releasable locking mechanism is locked in place to secure the frictional engagement of the water pipe by the first and second jaws and wherein the releasable locking mechanism is unlocked and a separation between the first jaw and the second jaw is increased for frictionally disengaging of the water pipe.

3. A method and device for vaporizing phyto material according to claim 1 wherein the frictional engagement mechanism comprises an adjustable clamping mechanism comprises a first jaw and a second jaw disposed opposite the first jaw, the first and second jaws mechanically coupled to a lead screw, for upon rotating of the lead screw in a clockwise direction for increasing a frictional engagement of the water pipe and for upon rotating of the lead screw in a counter clockwise direction for decreasing a frictional engagement of the water pipe, wherein a spacing between the first jaw the first and second jaws and for respectively sliding within a first track and a second track.

4. A method and device for vaporizing phyto material according to claim 3 comprising a motor mechanically coupled to the lead screw and electrically coupled with the first control circuit for controllably rotating of the lead screw for rotating in a clockwise direction for increasing the frictional engagement of the water pipe and for upon rotating of the lead screw in the counter clockwise direction for decreasing the frictional engagement of the water pipe.

5. A method and device for vaporizing phyto material according to claim 2 comprising:
a twist lock coupling having rotating portion and a static portion, the twist lock coupling rotating portion coupled with the adjustable clamping mechanism and the static portion coupled with the first housing, the twist lock coupling for operating in locked mode of operation and an unlocked mode of operation, in the locked mode of operation the rotating portion and the static portion are frictionally engaged together and the water pipe is coupled with the first housing and in the unlocked mode of operation the rotating portion and a static portion are other than frictionally engaged and the water pipe is un coupled with the first housing;
wherein the rotating portion of the twist lock coupling is for first frictionally engaging with the water pipe through the adjustable clamping mechanism so that it is securely engaged with the water pipe in the locked mode of operation.

6. A method and device for vaporizing phyto material according to claim 1 wherein the frictional engagement mechanism comprises:
an adjustable clamping mechanism comprises a first jaw and a second jaw disposed opposite the first jaw, the first and second jaws mechanically coupled to a lead screw, for upon rotating of the lead screw in a clockwise direction for increasing a frictional engagement of the water pipe and for upon rotating of the lead screw in a counter clockwise direction for decreasing a frictional engagement of the water pipe, wherein a spacing between the first jaw the first and second jaws and for respectively sliding within a first track and a second track;
a twist lock coupling having rotating portion and a static portion, the twist lock coupling rotating portion coupled with the adjustable clamping mechanism and the static portion coupled with the first housing, the twist lock coupling for operating in locked mode of operation and an unlocked mode of operation, in the locked mode of operation the rotating portion and the static portion are frictionally engaged together and the water pipe is coupled with the first housing and in the unlocked mode of operation the rotating portion and a static portion are other than frictionally engaged and the water pipe is un coupled with the first housing,
wherein the rotating portion of the twist lock coupling is for first frictionally engaging with the water pipe through the adjustable clamping mechanism so that it is securely engaged with the water pipe and the water pipe with the rotating portion is inserted into the static portion that is coupled with the first housing and twisted into place with a rotation in a first direction to initiate the locked mode of operation.

7. A method and device for vaporizing phyto material according to claim 1 comprising:
a second control circuit disposed as part of the electronic vaporization element (EVE) and electrically coupled with the first and second electrical contacts, of the resistive heater and having a power coupling input port and a second wireless transceiver;
a first control circuit disposed within the first housing and electrically coupled with the electrical power source and comprising a power coupling output port and a first wireless transceiver; and,
an electronic vaporization element coupling cable for electrically coupling of the first control circuit to the second control circuit and the first wireless transceiver for communicating with the second wireless transceiver through a wireless communication link, whereby the electronic vaporization element coupling cable provides electrical power to the second control circuit and the wireless communication link is for exchanging a control data between the first and second control circuits and for at least one of heating and maintaining of the heating element at the predetermined temperature.

8. A method and device for vaporizing phyto material according to claim 5 wherein the first wireless transceiver comprises a first optical transceiver and the second wireless transceiver comprises a second optical transceiver as the wireless communication link wherein the control data between the first and second control circuits and is transmitted optically for at least one of heating and maintaining of the phyto material at the predetermined temperature and disabling operation of the heating element.

9. A method and device for vaporizing phyto material according to claim 1 comprising:
a first temperature sensor in thermal communication with the heating element, the first temperature sensor comprising a temperature signal output port and for generating a temperature signal in dependence upon a temperature of the heating element 8806;
an electronic vaporization element coupling cable for electrically coupling of the first control circuit to the heating element first electrical contact and a second electrical contact and the temperature signal output port, wherein the first control circuit is for receiving of the temperature signal and for pulse width modulating electrical power provided to the resistive heater along the electronic vaporization element coupling cable from the electrical power source for at least one of heating and maintaining of the phyto material at the predetermined temperature and disabling operation of the heating element.

10. A method and device for vaporizing phyto material according to claim 1 wherein the elongated hollow member comprises:
a phyto material contact surface disposed between the resistive heater and the phyto material, the phyto material contact surface for receiving of thermal energy from the resistive heater on a second side thereof and for transmitting at least a portion of the receive thermal energy into the phyto material disposed on the phyto material contact surface for the at least one of heating and maintaining of the phyto material at the predetermined temperature.

11. A method and device for vaporizing phyto material according to claim 10 wherein the phyto material contact surface comprises glass and the resistive heater comprises a ceramic heater, where the ceramic heater heats the phyto material through the glass phyto material contact surface where the phyto material does not contact the ceramic heater directly.

12. A method and device for vaporizing phyto material according to claim 10 wherein the phyto material contact surface comprises glass and the elongated hollow member comprises glass and the phyto material contact surface is disposed proximate the first end, wherein the heating element is releasably coupled with the elongated hollow member proximate the first end using a frictional coupling.

13. A method and device for vaporizing phyto material according to claim 10 wherein the phyto material contact surface comprises ceramic and the elongated hollow member comprises ceramic and the phyto material contact surface is disposed proximate the first end, wherein the heating element is releasably coupled with the elongated hollow member proximate the first end.

14. A method and device for vaporizing phyto material according to claim 1 comprising:
a first temperature sensor disposed proximate the heating element and the second side of phyto material contact surface and in thermal communication therewith, the temperature sensor comprising a temperature signal output port for generating a temperature signal, the first control circuit for generating a first temperature signal data from the temperature signal in dependence upon a temperature of the heating element,
wherein the first control circuit comprises a first lookup table, wherein the first lookup table comprises at least a calibration value for determining the predetermined temperature with the first temperature signal data.

15. A method and device for vaporizing phyto material according to claim 14 comprising:
a second temperature sensor electrically coupled with the second control circuit, the second temperature sensor for measuring a temperature of the ambient air, wherein the temperature of the ambient air along with the temperature signal data is used for determining the predetermined temperature.

16. A method and device for vaporizing phyto material according to claim 1 comprising a LED electrically coupled with first control circuit and protruding has the first housing for illuminating of the water pipe.

17. A method and device for vaporizing phyto material according to claim 1 comprising a LED display comprising a plurality of three color light emitters arranged in a two dimensional matrix for being electrically coupled with first control circuit for illuminating the water pipe and for the light to be reflected and refracted by the water pipe.

18. A method and device for vaporizing phyto material according to claim 1 wherein the frictional engagement mechanism comprises at least one of a suction cup device wherein the suction cup device is for use in frictionally engaging of the water pipe through formation of an at least partial vacuum between the suction cup device and the water pipe and an adhesive tape for adhering of the water pipe to the first housing.

19. A method and device for vaporizing phyto material according to claim 1 comprising:
a voice recognition processor comprising one of an Alexa Voice Services (AVS) and a Google® Home Voice Services one of electrically and wirelessly coupled with the first control circuit, the voice recognition processor for receiving of voice commands from a user for at least one of controlling the heating of the phyto material extracts to the predetermined temperature and for adjusting of the predetermined temperature and disabling operation of the heating element.

20. A method and device for vaporizing phyto material according to claim 1 comprising:
a cavity formed within the first housing for receiving of the voice recognition processor therein, wherein the voice recognition processor comprises at least one LED and the at least one LED is for illuminating of at least a portion of the water pipe through optical reflection and refraction.

21. A method and device for vaporizing phyto material according to claim 1 wherein the first control circuit comprises at lease one of a WIFI module electrically coupled therewith for communicating with the internet for at least one controlling the heating of the phyto material to the predetermined temperature and for adjusting of the predetermined temperature through the internet and a Bluetooth® module for communicating with a smartphone having a smartphone application for being executed therein, wherein the smartphone application communicates with the first control circuit through the Bluetooth® module for at least one controlling the heating of the phyto material to the predetermined temperature and for adjusting of the predetermined temperature and disabling operation of the heating element.

22. A method and device for vaporizing phyto material according to claim 1 comprising a speaker disposed within the first housing, the speaker electrically coupled with the first control circuit.

23. A method and device for vaporizing phyto material according to claim 1 comprising a tilt sensor electrically coupled with the first control circuit for determining whether the first housing has become inverted and for disabling the heating element for heating the phyto material to the predetermined temperature.

24. A method and device for vaporizing phyto material according to claim 1 comprising:
a syringe for being filled with the phyto material extract having a phyto material extract output port;
a syringe actuator electrically coupled with the first control circuit for actuating the syringe for depositing a predetermined volume of the phyto material extract onto the phyto material contact surface from the phyto material extract output port;
an ambient air input aperture for receiving of the ambient air prior to it contacting the first end of the elongated member;
a mass airflow meter in fluid communication with the first end of the elongated member and disposed downstream of the ambient air input aperture, the mass airflow meter for measuring mass of ambient air passing therethrough per unit of time and for generating a first air mass signal in dependence upon an initial flow of ambient air passing therethrough and for generating a first air mass data based on the mass of air passing therethrough and electrically coupled with the first control circuit, the first control circuit for processing of the first air mass data and for at least one of controlling of the predetermined volume of the phyto material extract being deposited per unit of time onto the phyto material contact surface and for controlling of the predetermined temperature of the phyto material contact surface.

25. A method and device for vaporizing phyto material according to claim 1 comprising:
a syringe for being filled with the phyto material extract having a phyto material extract output port;
a syringe actuator electrically coupled with the first control circuit and mechanically coupled with the syringe for actuating the syringe for depositing a predetermined volume of the phyto material extract onto the phyto material contact surface from the phyto material extract output port 4200a;
a robotic arm comprising an end effector and electrically coupled with the first control circuit and coupled with the syringe actuator, the robotic arm for controllably positioning of the phyto material extract output port proximate the phyto material contact surface for depositing a predetermined volume of the phyto material extract onto the phyto material contact surface.

26. A method and device for vaporizing phyto material for frictionally engaging a water pipe having a water pipe input port and an inhalation aperture with a water pipe fluid pathway formed therebetween comprising:
a electronic vaporization element (EVE) comprising:
an elongated hollow member comprising a low thermally conductivity material having a first end and a second end opposite the first end, a fluid pathway propagating from the first end to the second end thereof, the second end for coupling with the water pipe input port, the elongated hollow member proximate the first end having a phyto material contact surface and having disposed opposite thereof a second side phyto material contact surface, the phyto material for being applied to the phyto material contact surface proximate the first end;
a heating element comprising a first electrical contact and a second electrical contact and disposed proximate the first end and in proximity of the second side phyto material contact surface and opposite the phyto material contact surface, the heating element being partially disposed within a heating element housing;
a frictional coupling formed between the heating element housing and the elongated hollow member proximate the first end for releasably coupling of the heating element with the elongated hollow member proximate the first end;
the heating element for applying heat to the second side phyto material contact surface and for a portion of the applied heat to propagate through the elongated hollow member proximate the first end into the phyto material contact surface to which the phyto material is applied, the phyto material contact surface for heating of the phyto material by the propagated portion of the applied heat to a predetermined temperature for vaporizing of the phyto material for creating a vapor therefrom and upon inhalation from the inhalation aperture this vapor is mixed with ambient air and flows through the fluid pathway from the first end and propagates through the input port of the water pipe and through to the inhalation aperture;
a removable electrical power source comprising a first housing for having an electrical power source contained therein, the first housing comprising a frictional engagement mechanism for frictionally engaging of the water pipe; and,
a first control circuit disposed within the first housing and electrically coupled with the electrical power source and the first and second electrical contacts of the EVE with an electronic vaporization element coupling cable electrically disposed therebetween, the first control circuit for providing electrical power from the electrical power source to the heating element for heating the phyto material to the predetermined temperature.

27. A method and device for vaporizing phyto material for frictionally engaging a water pipe having an input port and an inhalation aperture with a water pipe fluid pathway formed therebetween comprising:
providing an electronic vaporization element (EVE) comprising an elongated hollow member having a first end disposed proximate a heating element and a second end opposite the first end, a fluid pathway propagating from the first end to the second end thereof with the heating element disposed proximate the first end;
coupling the EVE second end with the water pipe input port;
providing a first housing for having an electrical power source contained therein and comprising a frictional engagement mechanism for releasably frictionally engaging the water pipe;
frictionally engaging the water pipe with the frictional engagement mechanism for releasably coupling of the first housing to the water pipe;
disposing phyto material extract proximate the heating element;
heating of the phyto material extract to a predetermined temperature, where the predetermined temperature is a temperature that results in a vaporization of the phyto material;
vaporizing of the phyto material extract for creating a vapor therefrom; and
inhaling from the inhalation aperture and having the vapor mixing with ambient air for flowing through the fluid pathway from the first end through the second end and through the input port of the water pipe and through to the inhalation aperture.
